(19) United States
(12) Patent Application Publication
(10) Pub. No.: US 2025/0134949 A1
(43) Pub. Date: May 1, 2025

(54) PROMININ-1 PEPTIDE FOR TREATING LUNG INJURY

(71) Applicant: Children's Medical Center Corporation, Boston, MA (US)

(72) Inventors: Avner Adini, Brookline, MA (US); Benjamin Matthews, West Newton, MA (US)

(73) Assignee: Children's Medical Center Corporation, Boston, MA (US)

(21) Appl. No.: 18/741,486

(22) Filed: Jun. 12, 2024

Related U.S. Application Data
(63) Continuation of application No. 16/617,781, filed on Nov. 27, 2019, now abandoned.
(60) Provisional application No. 62/513,971, filed on Jun. 1, 2017.

(51) Int. Cl. A61K 38/10; A61K 9/00; A61K 47/14; A61P 11/00; C07K 14/475

(52) U.S. Cl. CPC A61K 38/10; A61K 47/14; A61P 11/00; C07K 14/475; A61K 9/0019

(57) ABSTRACT

Described herein are compositions and methods for treating a lung disorder associated with dysregulated VEGF signaling. The PR1P peptide (DRVQRQTTTVVA, SEQ ID NO: 1) and variants thereof are able to enhance VEGF signaling in the lungs and reduce lung cell apoptosis (e.g., induced by toxicity or injury), thus treating the disorder.

### RELATED APPLICATIONS

This application claims the benefit of the filing date under 35 U.S.C. § 119 of U.S. Provisional Application Ser. No. 62/513,971, filed Jun. 1, 2017, and entitled PROMININ-1 PEPTIDE FOR TREATING LUNG INJURY, the entire contents of which are incorporated herein by reference.

### BACKGROUND

Vascular Endothelial Growth Factor (VEGF) expresses at 500-fold higher concentration in the lungs compared to serum and plays a significant role in maintaining lung tissue homeostasis. Dysregulation in VEGF signaling has been implicated in various lung disorders.

### SUMMARY

Described herein are compositions and methods for treating a lung disorder associated with dysregulated VEGF signaling. A previously described angiogenic 12 amino-acid peptide (PR1P) that binds VEGF and increases VEGF binding to VEGF receptors and to endothelial cells in vitro is shown herein to upregulate VEGF signaling in endothelial cells in-vitro and in murine lung cells in vivo following administration (e.g., by inhalation). In addition, PR1P reduced lung cell apoptosis induced by injury or toxicity in vitro and in vivo.

Accordingly, some aspects of the present disclosure provide methods of treating a lung disorder associated with dysregulated VEGF signaling, the method comprising administering to a subject in need thereof an effective amount of a peptide comprising an amino acid sequence that is at least 80% or at least 90% identical to the amino acid sequence of DRVQRQTTTVVA (SEQ ID NO: 1).

In some embodiments, the peptide is no more than 50 amino acids in length. In some embodiments, the peptide comprises the amino acid sequence of SEQ ID NO: 1. In some embodiments, the peptide consists of the amino acid sequence of SEQ ID NO: 1. In some embodiments, the peptide comprises an amino acid sequence that has one or more conservative amino acid substitutions in SEQ ID NO: 1.

In some embodiments, the peptide is cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combinations thercof.

In some embodiments, the peptide further comprises a fusion domain. In some embodiments, the fusion domain is selected from the group consisting of polyhistidine, Glu-Glu, glutathione S transferase (GST), thioredoxin, protein A, protein G, an immunoglobulin heavy chain constant region (Fc), maltose binding protein (MBP), and human serum albumin. In some embodiments, the Fc is from human IgG1.

In some embodiments, the peptide is a dimer, trimer, tetramer, or pentamer.

In some embodiments, the peptide is attached to a polymer. In some embodiments, the polymer prolongs serum half-life of the peptide. In some embodiments, the polymer prolongs shelf-life of the peptide.

In some embodiments, the peptide is a cyclic peptide.

In some embodiments, the peptide is formulated in a pharmaceutical composition. In some embodiments, the pharmaceutical composition further comprises a pharmaceutically acceptable carrier.

In some embodiments, the peptide stabilizes VEGF. In some embodiments, the peptide prevents VEGF from proteolytic degradation. In some embodiments, the peptide upregulates VEGF signaling. In some embodiments, the peptide reduces lung cell apoptosis.

In some embodiments, the lung disorder is selected from the group consisting of: severe progressive pulmonary hypertension (PH), neonatal respiratory distress syndrome (RDS), scleroderma with interstitial lung disease, ARDS, COPD, emphysema and bronchopulmonary dysplasia (BPD).

In some embodiments, the lung disorder is associated with cigarette smoke. In some embodiments, the lung disorder is caused by LPS. In some embodiments, the lung disorder is associated with acute or chronic lung injury. In some embodiments, the lung disorder is emphysema. In some embodiments, the lung disorder is chronic obstructive pulmonary disease (COPD).

In some embodiments, the peptide is administered systemically. In some embodiments, the peptide is administered via intravenous injection. In some embodiments, the peptide is administered directly to the lung. In some embodiments, the peptide is administered via inhalation or instillation. In some embodiments, the peptide is administered repeatedly.

In some embodiments, administering a second agent to the subject in need thereof for the treatment of the lung disorder.

In some embodiments, the subject in need thereof is a mammal. In some embodiments, the mammal is a human. In some embodiments, the mammal is a rodent. In some embodiments, the rodent is a mouse or a rat.

Further provided herein are peptides comprising an amino acid sequence that is at least 80% or at least 90% identical to the amino acid sequence of DRVQRQTTTVVA (SEQ ID NO: 1), for use in the manufacturing of a medicament for treating a lung disorder associated with dysregulated VEGF signaling.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIGS. 1A to 1F. PR1P increases VEGF signaling in lung epithelial cells in vitro and in lung cells in vivo. (FIG. 1A) Representative FACS analysis of BEAS-2B cells following incubation with VEGF, PR1P or scrambled peptide (SP) showing increased phosphorylation of VEGFR2 by PR1P compared to SP. (FIG. 1B) Quantification of VEGFR phosphorylation from FACS experiments described in FIG. 1A. Data are mean±SEM (n=3). P<0.02. (FIG. 1C-1D) Representative western blot (non-reduced gel, FIG. 1C) and densitometric quantification of blots (FIG. 1D) of VEGFR and AKT phosphorylation in lysates of BEAS-2B cells treated as described in FIG. 1A. Data in FIG. 1D are mean±SEM (n=3). P AKT p=0.0209, pVEGFR-2 p=0.05. (FIG. 1E) Representative FACS analysis of cells harvested from mouse lungs following inhalation of nebulized PR1P, SP or VEGF showing increased pVEGFR2 following VEGF or PR1P inhalation. (FIG. 1F) Quantification of pVEGFR2 from FACS experiments described in FIG. 1E. Data are mean±SEM (n=3). P<0.03.

FIGS. 2A to 2G. PR1P reduces cigarette smoke induced lung epithelial cell apoptosis in vitro. (FIG. 2A) Phase microscope images of BEAS-2B cells prior to (0 h) and 24 h following exposure to cigarette smoke treated media in the absence and presence of decreasing concentrations of PR1P showing that PR1P reduces proportion of smoke induced round cells (flat and round cells highlighted in FIG. 2B. (FIG. 2C) Quantification of the proportion of round cells compared to total cells in experiments described in FIG. 2A. Data are mean±SEM (n=3). P<0.05. (FIG. 2D) PR1P mitigates morphometric changes in BEAS-2B cells caused by cigarette smoke exposure. Identical areas of BEAS-2B cells (1×10⁵) cultured for 2 days on six well plates in DMEM containing 10% serum were imaged using phase microscopy prior to (0 h) and 24 hours (24 h) following exposure to fresh serum free media or cigarette smoke treated serum-free media in the absence and presence of decreasing concentrations of PR1P. Image analysis showed that cigarette smoke exposure induced a decrease in the proportion of spread cells (FIG. 2D) that were mitigated by PR1P (*p<0.005 using t-test vs PR1P groups). (FIG. 2E) Merged phase and fluorescence images of cells treated as in FIG. 2A and stained after 24 hours with TUNEL to identify apoptosis showing that round cells are apoptotic. (FIG. 2F) Representative FACS analysis of cells treated as described in FIG. 2A and stained with TUNEL showing PR1P reduces smoke induced apoptosis. (FIG. 2G) Quantification of apoptosis (TUNEL mean Fluorescence Intensity (MFI)) from FACS experiments described in FIG. 2F. Data are mean±SEM (n=3). P<(*0.05 ** 0.01).

FIGS. 3A to 3F. Inhaled PR1P reduces LPS induced lung cell apoptosis in-vivo. (FIG. 3A-3B) Representative FACS analyses of cells harvested from the lungs of mice 24 h after treatment with nebulized LPS+SP or LPS+PR1P showing PR1P reduces cell apoptosis identified using Capase-3 (FIG. 3A) and Annexin-V markers (FIG. 3B) P<0.05. (FIGS. 3C-3D) Quantification of FACS experiments described in FIGS. 3A-3B using Caspase-3 (FIG. 3C) or Annexin-V (FIG. 3D) as markers of apoptosis. Data are mean±SEM (n=4). *p<0.02. (FIG. 3E) Representative FACS analysis of lung epithelial cells harvested from the lungs of mice treated as described in FIG. 3A showing PR1P reduces lung epithelial cell apoptosis identified using Capase-3 and anti-CD326 antibody (to identify epithelial cells). (FIG. 3F) Quantification of FACS experiments described in FIG. 3E. Data are mean±SEM (n=6?). *p<0.03.

FIGS. 4A to 4F. Dot blot analysis and 3D computational modeling and docking simulation data suggest that PR1P competes with plasmin and elastase binding to the VEGF heparin binding domain (HBD). (FIG. 4A) PR1P protects VEGF from protease degradation as shown by a VEGF peptide array Prominin-1 binding assay. (FIG. 4A) Four 12-mer VEGF-derived peptides (VP1-VP4) displaying the greatest binding of Prominin-1 (amongst 179 peptides tested) were each derived from the VEGF heparin binding domain (HBD) of VEGF, as shown in FIG. 4B. (FIG. 4B) A schematic representation of two 165 amino acid VEGF monomers linked by two disulfide bonds (s-s) forming a VEGF₁₆₅ heterodimer. The blue regions represent the amino-terminal (N) amino acids 1-110 and the red regions represent the HBD carboxy-terminal (C) amino acids 111-165. The plasmin cleavage sites are indicated by green arrows (between amino acids 109 (R) and 110 (FIG. 4A). The amino acid sequences 106-109 (blue font) and the entire HBD (110-165, red font) are shown along with the location and sequences of the four overlapping 12-mer VEGF peptides VP1-VP4 (grey boxes described in FIG. 4A). (FIG. 4C) Simulation of Prominin-1 extracellular fragment binding to VEGF-HBD fragment. FIG. 4C shows a close-up view of the interaction residues between prominin-1 and VEGF (HBD). Residues E341, D352, D354, Y361, and Q372 are from prominin-1 (blue) while residues R2, N5, R14, K15, R35, R39, R49, and R55 are from VEGF (green). The H-bonds are indicated in red. (FIG. 4D) Simulation of PR1P binding to VEGF-HBD fragment. FIG. 4D shows a ribbon representation of the 3D molecular modeling of complex of PR1P (green) and VEGF(HBD) (blue). The H-bonds are indicated in red. (FIG. 4E) Simulation of plasmin and elastase binding to the VEGF-HBD (FIG. 4F) Alteration of the amino acid D to A reduces dramatically the binding of VEGF to endothelial cells.

FIG. 5. Representative western blot (non-reduced gel) analysis of VEGF protein incubated (2 h) in the absence or presence of the proteases plasmin (left gel) or elastase (right gel) in the absence or presence of PR1P showing that PR1P reduces the proportion of protease induced VEGF degradation products.

FIGS. 6A to 6D. Schematic of experimental summary and proposed PR1P enhanced VEGF signaling. (FIG. 6A) Three 12-mer peptides whose amino acid sequences were derived from Prominin-1 that displayed high affinity for VEGF were each derived from one of Prominin-1's five extracellular domains (sec FIGS. 1A-1F). Note that PR1P, a 12-mer peptide with the highest binding affinity for VEGF is depicted as a large black dot. (FIG. 6B) Four 12-mer peptides whose amino acid sequences were derived from VEGF that displayed high affinity for Prominin-1 were each derived from sequences within the HBD of VEGF (see FIG. 5). Together these findings suggest that PR1P binds to VEGF on or near the HBD. (FIGS. 6C-6D) A covalently linked VEGF dimer binds to a VEGF receptor monomer (FIG. 6C) leading to dimerization of two receptor monomers (VEGFR, FIG. 6D) which leads to autophosphorylation of the dimerized VEGFR(60) (p-VEGFR), phosphorylation of AKT (p-AKT), and VEGF mediated survival signaling through AKT. In the presence of proteases, VEGF is cleaved into VEGF degradation products with altered VEGF receptor binding properties (data not shown). PR1P binds to VEGF near or on the HBD, blocks VEGF cleavage by proteases, and stabilizes VEGF leading to increased VEGF binding to, and affinity for VEGFR, leading to increased autophosphorylation of VEGFR, phosphorylation of AKT, and improved cell survival (data not shown).

FIG. 7. PR1P neither mitigates cigarette smoke induced arrest of BEAS-2B cell proliferation or acute cell death. Quantification of BEAS-2B cells before and after exposure on day 2 (arrow) to Cigarette Smoke Exposed (Smoke) media in the absence (Control) or presence of PR1P. Data are mean±SEM (n=3).

FIG. 8. Inhaled PR1P reduces LPS induced neutrophil migration into lungs. Representative FACS analysis of cells harvested from mouse lung 24 h after treatment with nebulized LPS+SP or LPS+PR1P showing that PR1P reduces migration of inflammatory cells (by 2 folds) to the lungs (top) that can be identified as neutrophils (bottom).

FIGS. 9A to 9D. Smoke exposure method. A single cigarette (Marlboro) is lit and burned for 3 minutes underneath an open and upturned 50 mL Falcon tube (FIG. 9A). The Falcon tube is capped and filled with 30 mL DMEM (FIG. 9B). Smoke exposed media is then filtered (22 μm, FIG. 9C) and used for experiments as described (FIG. 9D).

FIG. 10. FACS analysis confirms that PR1P protects BEAS-2B cells from cell apoptosis caused by cigarette smoke exposure. BEAS-2B cells (1×10⁵) plated for 24 h on six well plates in DMEM containing 10% serum were exposed to fresh serum-free media (Control) or cigarette smoke treated serum-free media (Smoke) for 24 hours in the absence and presence of decreasing concentrations of PR1P, trypsinized and prepared for FACS analysis using a standard TUNEL staining method to identify apoptosis. Values highlighted by squares in each condition indicate percentage of positively stained cells (i.e. apoptotic cells). Note that cigarette smoke exposure induced an increase in the proportion of apoptotic cells compared to control that was mitigated by PR1P. Data shown are representative of two independent experiments.

FIG. 11. Elastase induced murine emphysema phenotype was evident at 24 h and was indistinguishable at 24 h from injury seen at 4 days. This figure shows representative photomicrographs of H&E stained lung sections obtained from mice 24 h after intratracheal treatment with normal saline (left panel) or elastase (middle panel), or 4 days after intra-tracheal elastase (right panel). Typical features of emphysema including enlarged alveoli and destruction of alveolar walls. These injuries were evident at 24 h and were indistinguishable from discasc at 4 days.

FIGS. 12A-12D. Inhaled PR1P improved lung architecture after elastase induced emphysema in mice. (FIG. 12A) Representative photomicrographs of H&E stained lung sections obtained from mice 4 days after intra-tracheal injections of normal saline (left panel), or elastase followed by daily treatment of inhaled normal saline (middle panel) or PR1P (right panel). (FIG. 12B) Bar graph showing combined results of blinded qualitative analysis of lung emphysema injury score assessments of whole lung sections from experiments described in FIG. 12A. The results indicate significant reduction in emphysema phenotype by inhaled PR1P (n=4 experiments, 12 mice total per group, p<0.001). (FIG. 12C) Representative analysis of quantification of line segment lengths from randomly sampled images from of mouse lungs from experiments described in FIG. 12A. Typical increase in line segment length induced by intratracheal elastase treatment was reduced at day 4 by daily inhalation treatment with PR1P. Data were representative of n=4 experiments with 12 mice total in each group, p<0.001. (FIG. 12D) Representative linear scale distribution analysis of line segment lengths obtained from analysis of a single experiment described in FIG. 12B. Elastase induced emphysema was associated with a wider and lower peak in the distribution curve. The curve was narrower and higher with PR1P treatment. Data were representative of n=4 experiments with 12 mice total in each group.

FIGS. 13A-13B. Treatment of lung epithelial cells in vitro with PR1P resulted in VEGFR2 phosphorylation. (FIG. 13A) Representative FACS analysis of BEAS-2B cells following incubation with PR1P in the presence or absence of the VEGFR2 inhibitor SU5416 show that increased VEGFR2 phosphorylation induced by PR1P (top right) was abrogated in the presence of SU5416 (bottom right). Numbers in the 4 corners are percentage of cells in each quadrant. (FIG. 13B) Paired raw data from individual experiments showing changes in VEGFR2 phosphorylation levels in FACS experiments described in FIG. 13A (n=4, p<0.02).

FIGS. 14A-14B. Upregulation of downstream VEGF signaling by PR1P requires activation of VEGFR2. (FIG. 14A) Representative FACS analysis of BEAS-2B cells following incubation with PR1P in the presence or absence of the VEGFR2 inhibitor SU5416 showing that increased AKT phosphorylation induced by PR1P (top right) was abrogated in the presence of SU5416 (bottom right). Numbers in the four corners are percentage of cells in each quadrant. (FIG. 14B) Paired raw data from individual experiments showing changes in AKT phosphorylation levels from FACS experiments described in FIG. 14A (n=4, p<0.02).

### DETAILED DESCRIPTION OF CERTAIN EMBODIMENTS

Vascular Endothelial Growth Factor (VEGF) mediates cell survival and apoptosis pathways via binding to and activation of Vascular Endothelial Growth Factor receptor-2(VEGFR-2) and signaling through the PI3K/AKT pathway. The present disclosure is based, at least in part, on the findings that a previously described 12-amino acid peptide derived from an extracellular VEGF binding domain Prominin-1 (termed herein as “PR1P”, DRVQRQTTTVVA (SEQ ID NO: 1)) enhances VEGF signaling and reduces apoptosis in cells in vitro and in murine model. PR1P has previously been shown to bind VEGF, increase VEGF binding to VEGF receptors VEGFR2 and Neuropilin-1 and to endothelial cells in vitro, and increase VEGF dependent angiogenesis in multiple murine angiogenesis models in vivo (e.g., as described in Adini et al., Angiogenesis (2017), doi: 10.1007/s10456-017-9556-7,and International Patent Application Publication WO2010014616, and International Patent Application Publication WO2011094430A2, incorporated herein by reference). Without wishing to be bound by scientific theory, the efficacy of PR1P in treating a lung disorder associated with dysregulated VEGF signaling is believed to be based on its effects in reducing apoptosis of the cells that have dysregulated VEGF signaling, and is independent of the angiogenic effects of VEGF described in the aforementioned references.

Accordingly, some aspects of the present disclosure relate to methods of treating a lung disorder associated with dysregulated VEGF signaling, the method comprising administering to a subject in need thereof an effective amount of a peptide comprising an amino acid sequence that is at least 80% identical to the amino acid sequence of DRVQRQTTTVVA (SEQ ID NO: 1).

In some embodiments, the peptide used in the methods described herein comprises an amino acid sequence that is at least 80% or at least 90% identical to the amino acid sequence of DRVQRQTTTVVA (SEQ ID NO: 1). In some embodiments, the peptide may comprise an amino acid sequence that is 80% identical or 90% identical to the amino acid sequence of DRVQRQTTTVVA (SEQ ID NO: 1). In some embodiments, the peptide comprises the amino acid sequence of SEQ ID NO: 1. In some embodiments, the peptide consists of the amino acid sequence of SEQ ID NO: 1.

In some embodiments, the peptide is 10-100 amino acids in length and comprises an amino acid sequence that is at least 80% identical to the amino acid sequence of DRVQRQTTTVVA (SEQ ID NO: 1). For example, the peptide may be 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 amino acids long and comprises an amino acid sequence that is at least 80% identical to the amino acid sequence of DRVQRQTTTVVA (SEQ ID NO: 1). In some embodiments, the peptide is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids long. In some embodiments, the peptide may be more than 100 amino acids long.

In some embodiments, the peptide comprises an amino acid sequence that is longer or shorter than SEQ ID NO: 1 and is at least 80% identical to the amino acid sequence of SEQ ID NO: 1. For example, the peptide may comprise an amino acid sequence that is 1 or 2 amino acids longer (addition) or shorter (truncation) than SEQ ID NO: 1 and is at least 80% identical to the amino acid sequence of SEQ ID NO: 1. The addition or truncation may be at either N- or C-terminal end of SEQ ID NO: 1 or at both N- and C-terminal ends of SEQ ID NO: 1.

In some embodiments, the peptide comprises an amino acid sequence that has one or more (e.g., 1 or 2) conservative amino acid substitutions at any position in SEQ ID NO: 1. A “conservative amino acid substitution” is an amino acid substitution that changes an amino acid to a different amino acid with similar biochemical properties (e.g. charge, hydrophobicity and size). Conservative substitutions of amino acids include, for example, substitutions made amongst amino acids within the following groups: (a) M, I, L, V; (b) F, Y, W; (c) K, R, H; (d) A, G; (e) S, T; (f) Q, N; and (g) E, D. Conservative amino acid substitutions do not alter the relative charge or size characteristics of the protein in which the amino acid substitutions are made. Conservative amino acid substitutions typically do not change the overall structure of the peptide and/or the type of amino acid side chains available for forming van der Waals bonds with a binding partner. In some embodiments, the peptide comprises an amino acid sequence that has one conservative amino acid substitution at any one of positions 1, 2, 3, 4, 5, 6, 7, 8, 9, 10, 11, 12 of SEQ ID NO: 1. In some embodiments, the peptide comprises an amino acid sequence that has two conservative amino acid substitutions at any two positions of positions 1, 2, 3, 4, 5, 6, 7, 8, 9, 10, 11, 12 of SEQ ID NO: 1.

Amino acid substitution can be achieved during chemical synthesis of the peptide by adding the desired substitute amino acid at the appropriate sequence in the synthesis process. Alternatively, molecular biology methods can be used. Non-conservative substitutions are also encompassed to the extent that they substantially retain the activities of those peptides described herein.

In some embodiments, the peptide comprises a modification. Peptides comprising modifications have additional features other than amino acid contents. As used herein, a “modification” or “derivative” of a peptide produces a modified or derivatized peptide, which is a form of a given peptide that is chemically modified relative to the reference peptide, the modification including, but not limited to, oligomerization or polymerization, modifications of amino acid residues or peptide backbone, cross-linking, cyclization, conjugation, pegylation, glycosylation, acetylation, phosphorylation, acylation, carboxylation, lipidation, thioglycolic acid amidation, alkylation, methylation, polyglycylation, glycosylation, polysialylation, adenylylation, fusion to additional heterologous amino acid sequences, or other modifications that substantially alter the stability, solubility, or other properties of the peptide while substantially retaining the activity of the peptides described herein. The peptide comprising the aforementioned modifications, are referred to as being cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combination thereof. As such, the peptides used in the methods described herein may contain non-amino acid elements, such as polyethylene glycols, lipids, poly-or mono-saccharide, and phosphates.

In some embodiments, the modification may be at the C-terminus (e.g., C-terminal amidation), N-terminus (e.g., N-terminal acetylation), or internally in the peptides used in the methods described herein. Terminal modifications reduce susceptibility to proteinase digestion, and therefore serve to prolong half-life of the peptides in solutions, particularly biological fluids where proteases may be present. In some embodiments, the peptides are further modified within the sequence, such as, modification by terminal-NH₂ acylation, e.g., acetylation, or thioglycolic acid amidation, by terminal-carboxylamidation, e.g., with ammonia, methylamine, and the like terminal modifications.

Terminal modifications are useful, to reduce susceptibility by proteinase digestion, and therefore can serve to prolong half-life of the peptides in solution, particularly in biological fluids where proteases may be present. Amino terminus modifications include methylation (e.g., —NHCH₃ or —N(CH₃)₂), acetylation (e.g., with acetic acid or a halogenated derivative thereof such as a-chloroacetic acid, a-bromoacetic acid, or a-iodoacetic acid), adding a benzyloxycarbonyl (Cbz) group, or blocking the amino terminus with any blocking group containing a carboxylate functionality defined by RCOO— or sulfonyl functionality defined by R—SO2—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide. In some embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides used in the methods described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art, for example, in U.S. Patent Application No. 20090035814; Muralidharan and Muir, 2006, Nat Methods, 3:429-38; and Lockless and Muir, 2009, Proc Natl Acad Sci U S A. June 18, Epub. C-terminal functional groups of the peptides used in the methods described herein include amide, amide lower alkyl, amide di(lower alkyl), lower alkoxy, hydroxy, and carboxy, and the lower ester derivatives thereof, and the pharmaceutically acceptable salts thereof.

In some embodiments, the peptides used in the methods described herein are phosphorylated. One can also readily modify peptides by phosphorylation, and other methods (e.g., as described in Hruby, et al. (1990) Biochem J. 268:249-262). One can also replace the naturally occurring side chains of the genetically encoded amino acids (or the stereoisomeric D amino acids) with other side chains, for instance with groups such as alkyl, lower (C1-6) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocycles. In some embodiments, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, pipcridino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl groups. These heterocyclic groups can be substituted or unsubstituted. Where a group is substituted, the substituent can be alkyl, alkoxy, halogen, oxygen, or substituted or unsubstituted phenyl.

In some embodiments, the peptides used in the methods of the present disclosure is multimeric, e.g., a dimer, trimer, tetramer, or pentamer. In some embodiments, the molecular linker used for forming the oligomeric peptides is a peptide linker molecule. In some embodiments, the peptide linking molecule comprises at least one amino acid residue which links at least two peptides according to the disclosure. The peptide linker comprises, e.g., at least 2, 3, 4, 5, 6, 7, 8, 9, 10 or more amino acids residues and preferably less than 50 amino acids residues. The peptide linking molecule can couple peptides or proteins covalently or non-covalently. Typical amino acid residues used for linking are glycine, tyrosine, cysteine, lysine, glutamic and aspartic acid, or the like. A peptide linker is attached on its amino-terminal end to one peptide, peptide or peptide domain (e.g., a C-peptide) and on its carboxyl-terminal end to another peptide, peptide or peptide domain (again, e.g., a C-peptide). Examples of useful linker peptides include, but are not limited to, glycine polymers ((G)n) including glycine-serine and glycine-alanine polymers (e.g., a (Gly4Ser) n repeat where n=1-8, preferably, n=3, 4, 5, or 6). Other examples of peptide linker molecules are described in U.S. Pat. No. 5,856,456 and are hereby incorporated by reference.

In some embodiments, the peptides used in the methods described herein are dimerized or multimerized by covalent attachment to at least one linker moiety. In some embodiments, the linker moiety is a C1-12 linking moiety optionally terminated with one or two —NH— linkages and optionally substituted at one or more available carbon atoms with a lower alkyl substituent. In some embodiments, the linker comprises —NH—R—NH— wherein R is a lower (C1-6) alkylene substituted with a functional group, such as a carboxyl group or an amino group, that enables binding to another molecular moiety (e.g., as may be present on the surface of a solid support during peptide synthesis or to a pharmacokinetic-modifying agent such as PEG). In some embodiments, the linker is a lysine residue. In some embodiments, the linker bridges the C-termini of two peptide monomers, by simultaneous attachment to the C-terminal amino acid of each monomer. In some embodiments, the linker bridges the peptides by attaching to the side chains of amino acids not at the C-termini. When the linker attaches to a side chain of an amino acid not at the C-termini of the peptides, the side chain may contain an amine, such as those found in lysine, and the linker contains two or morc carboxy groups capable of forming an amide bond with the peptides.

The peptides (e.g., monomers, dimers, or multimers) used in the methods described herein may be attached to one or more polymer moieties (e.g., covalently or non-covalently). In some embodiments, these polymers are covalently attached peptides. Preferably, for therapeutic use of the end product preparation, the polymer is pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations.

Suitable polymers include, without limitation, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof. Such a polymer may or may not have its own biological activity. The polymers can be covalently or non-covalently conjugated to the peptide. Methods of conjugation for increasing serum half-life are known in the art, for example, in U.S. Pat. Nos. 5,180,816, 6,423,685, 6,884,780, and 7,022,673, which are hereby incorporated by reference in their entirety.

In some embodiments, the polymer is a water soluble polymer such as, without limitation, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly (n-vinyl-pyrrolidone) polyethylene glycol, propropylene glycol homopolymers, polypropylene oxide/ethylene oxide copolymers, and polyox yethylated polyols. In some embodiments, the water soluble polymer is PEG.

The polymer may be of any molecular weight, and may be branched or unbranched. The average molecular weight of the reactant PEG is preferably between about 3,000 and about 50,000 daltons (the term “about” indicating that in preparations of PEG, some molecules will weigh more, and some less, than the stated molecular weight). More preferably, the PEG has a molecular weight of from about 10 kDa to about 40 kDa, and even more preferably, the PEG has a molecular weight from 15 to 30 kDa. Other sizes may be used, depending on the desired therapeutic profile (e.g., duration of sustained release desired; effects, if any, on biological activity; case in handling; degree or lack of antigenicity; and other effects of PEG on a therapeutic peptide known to one skilled in the art).

The number of polymer molecules attached may vary; for example, one, two, three, or more water-soluble polymers may be attached to a peptide of the disclosure. The multiple attached polymers may be the same or different chemical moieties (e.g., PEGs of different molecular weight).

In some embodiments, PEG may be attached to at least one terminus (N-terminus or C-terminus) of a peptide (i.e., the peptide is PEGylated). In other embodiments, PEG may be attached to a linker moiety to a peptide. PEGylation is routinely achieved by incubation of a reactive derivative of PEG with the target macromolecule. The covalent attachment of PEG to a peptide (e.g., a peptide drug) can “mask” the agent from the host's immune system (reduced immunogenicity and antigenicity), and increase the hydrodynamic size (size in solution) of the peptide which prolongs its circulatory time by reducing renal clearance. PEGylation can also provide water solubility to hydrophobic drugs and proteins. PEGylation, by increasing the molecular weight of a molecule, can impart several significant pharmacological advantages over the unmodified form, such as: improved drug solubility, reduced dosage frequency, without diminished efficacy with potentially reduced toxicity, extended circulating life, increased drug stability, and enhanced protection from proteolytic degradation. In addition, PEGylated drugs are have wider opportunities for new delivery formats and dosing regimens. Methods of PEGylating molecules, proteins and peptides are well known in the art, e.g., as described in U.S. Pat. Nos. 5,766,897; 7,610,156; 7,256,258 and the International Application No. WO/1998/032466.

The peptides used in the methods described herein can be conjugated to other polymers in addition to polyethylene glycol (PEG). The polymer may or may not have its own biological activity. Further examples of polymer conjugation include but are not limited to polymers such as polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof. Conjugation to a polymer can improve serum half-life, among other effects.

A variety of chelating agents can be used to conjugate the peptides used in the methods described herein. These chelating agents include but are not limited to ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA). Methods of conjugation are well known in the art, for example, P. E. Thorpe, et. al, 1978, Nature 271, 752-755; Harokopakis E., et, al., 1995, Journal of Immunological Methods, 185:31-42; S. F. Atkinson, et, al., 2001, J. Biol. Chem., 276:27930-27935; and U.S. Pat. Nos. 5,601,825, 5,180,816, 6,423,685, 6,706,252, 6,884,780, and 7,022,673, which are hereby incorporated by reference in their entirety.

In some embodiments, the peptides used in the methods described herein further comprises one or more fusion domains. Well known examples of such fusion domains include, without limitation, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel-or cobalt-conjugated resins are used. Many of such matrices are available in “kit” form, such as the Pharmacia GST purification system and the QIAexpress™ system (Qiagen) useful with (HIS6) fusion partners. In some embodiments, the peptide is fused with a domain that stabilizes the peptide in vivo (a “stabilizer” domain). “Stabilizing”, as used herein, means an increase in the half-life of the peptide in vivo, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains. In some embodiments, the peptides used in the methods described herein further comprises an Fc portion of human IgG1 (SEQ ID NO: 2).

Fc portion of human IgG1
(SEQ ID NO: 2)
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPVPIEKTISKAKGQPREPQVYTLPPSREEMTKNQVSLTCLVKGF
YPSDIAVEWESNGQPENNYKTTPPVLDSDGPFFLYSKLTVDKSRWQQGNV
FSCSVMHEALHNHYTQKSLSLSPGK

In some embodiments, attaching the peptide to a polymer or fusing the peptide to a fusion domain prolongs the serum half-life of the peptide. The “serum half-life” of a peptide refers to the period of time required for the concentration or amount of the peptide in the body to be reduced by one-half. A peptide's serum half-life depends on how quickly it is eliminated from the serum. The longer the serum half-life is, the more stable the peptide is in the body. “Prolongs serum half-life” means that when the peptide is attached to a polymer or fused to a fusion domain, the serum half-life of the peptide increases by at least 30%, compared to the peptide alone. For example, the serum half-life of the peptide may increase by at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold, or more, when the peptide is attached to a polymer or fused to a fusion domain, compared to the peptide alone. In some embodiments, the serum half-life of the peptide may increase by 30%, 40%, 50%, 60%, 70%, 80%, 90%, 100%, 2-fold, 3-fold, 4-fold, 5 fold- 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, or more, when the peptide is attached to a polymer or fused to a fusion domain, compared to the peptide alone.

In some embodiments, attaching the peptide to a polymer or fusing the peptide to a fusion domain prolongs the shelf-life of the peptide. The “shelf-life”, refers to the period of time, from the date of manufacture, that a product is expected to remain within its approved product specification while stored under defined conditions. It is desirable for a therapeutic agent, e.g., the peptides used in the methods of the present disclosure, to have a longer shelf-life. “Prolongs shelf-life” means that when the peptide is attached to a polymer or fused to a fusion domain, the shelf-life of the peptide increases by at least 30%, compared to the peptide alone. For example, the shelf-life of the peptide may increase by at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold, or more, when the peptide is attached to a polymer or fused to a fusion domain, compared to the peptide alone. In some embodiments, the shelf-life of the peptide may increase by 30%, 40%, 50%, 60%, 70%, 80%, 90%, 100%, 2-fold, 3-fold, 4-fold, 5 fold- 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, or more, when the peptide is attached to a polymer or fused to a fusion domain, compared to the peptide alone.

The peptide comprising the amino acid sequence of SEQ ID NO: 1 or a variant of SEQ ID NO: 1 (e.g., addition, truncation, amino acid substitution), or comprising any of the modification and/or derivations described herein substantially retain the activity of the peptide of SEQ ID NO: 1. By “substantially retain,” it means one or more activities of the peptide variant is at least 50% compared to the activities of the original peptide (SEQ ID NO: 1) in a similar assay, under similar conditions. For example, the activities of the peptide variants may be at least 50%, at least 60%, at least 70%, at least 80%, at least 90%, at least 95%, at least 99%, at least 100%, at least 2-fold, at least 5-fold, at least 10-fold, at least 100-fold or higher, compared to the original peptide (SEQ ID NO: 1).

In some embodiments, the peptide stabilizes VEGF. “Stabilizes VEGF” means that when the peptide is administered to the subject in need thereof, the half-life of VEGF (e.g., in the lungs of the subject) increases by at least 30%, compared to without the peptide. For example, the half-life of VEGF may increase by at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold, or more, when the peptide is administered to the subject in need thereof, compared to without the peptide. In some embodiments, the half-life of VEGF is increased by 30%, 40%, 50%, 60%, 70%, 80%, 90%, 100%, 2-fold, 3-fold, 4-fold, 5 fold-6-fold. 7-fold, 8-fold, 9-fold, 10-fold, 20-fold, 30-fold, 40-fold, 50-fold, 60-fold, 70-fold, 80-fold, 90-fold, 100-fold, 1000-fold, or more, when the peptide is administered to the subject in need thereof, compared to without the peptide.

In some embodiments, the peptide binds to VEGF and prevents VEGF from proteolytic degradation. Without wishing to be bound by scientific theory, certain lung disorder associated with dysregulated VEGF signaling also exhibits increased secretion of proteolytic enzymes, which degrades VEGF. It is shown herein that binding of the PR1P peptide to VEGF protects VEGF from proteolytic degradation. In some embodiments, the amount of VEGF that is protcolytically degraded is reduced by at least 30%, when the peptide is administered to the subject in need thereof, compared to without the peptide. For example, the amount of VEGF that is proteolytically degraded may be reduced by at least 30%, at least 40%, at least 50%, at least 60%, at least 70%, at least 80%, at least 90%, or 100% when the peptide is administered to the subject in need thereof, compared to without the peptide. In some embodiments, the amount of VEGF that is proteolytically degraded is reduced by 30%, 40%, 50%, 60%, 70%, 80%, 90%, or 100% when the peptide is administered to the subject in need thereof, compared to without the peptide.

In some embodiments, the peptides used in the methods described herein upregulates VEGF signaling. “Upregulate VEGF signaling” means that the magnitude of VEGF signaling is enhanced by at least 30% when the peptide is administered to the subject in need thereof, compared to without the peptide. For example, VEGF signaling may be upregulated by at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold, or more, when the peptide is administered to the subject in need thereof, compared to without the peptide. In some embodiments. VEGF signaling is upregulated by 30%, 40%, 50%, 60%, 70%, 80%, 90%, 100%, 2-fold, 3-fold, 4-fold, 5 fold- 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, or more, when the peptide is administered to the subject in need thereof, compared to without the peptide.

In some embodiments, the peptide reduces lung cell apoptosis. “Apoptosis” is a process of programmed cell death that occurs in multicellular organisms. Biochemical events lead to characteristic cell changes (morphology) and death, including blebbing, cell shrinkage, nuclear fragmentation, chromatin condensation, chromosomal DNA fragmentation, and global mRNA decay. It is shown herein that the PR1P peptide reduces lung cell apoptosis associated with injury and/or toxicity. “Reduce lung cell apoptosis” means that the number of lung cells that undergo apoptosis is reduced by at least 30% when the peptide is administered to the subject in need thereof, compared to without the peptide. For example, the amount of lung cells that undergo apoptosis may be reduced by at least 30%, at least 40%, at least 50%, at least 60%, at least 70%, at least 80%, at least 90%, or 100% when the peptide is administered to the subject in need thereof, compared to without the peptide. In some embodiments, the number of lung cells that undergo apoptosis is rcduccd by 30%, 40%, 50%, 60%, 70%, 80%, 90%, or 100% when the peptide is administered to the subject in need thereof, compared to without the peptide.

The peptides described herein may be formulated into pharmaceutical compositions. In some embodiments, the pharmaceutical composition further comprises a pharmaceutically acceptable carrier. The pharmaceutical composition can further comprise additional agents (e.g. for specific delivery, increasing half-life, or other therapeutic agents).

The term “pharmaceutically-acceptable carrier”, as used herein, means a pharmaceutically-acceptable material, composition or vehicle, such as a liquid or solid filler,

diluent, excipient, manufacturing aid (e.g., lubricant, talc magnesium, calcium or zinc stearate, or steric acid), or solvent encapsulating material, involved in carrying or transporting the peptide from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as peptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the peptides, or the composition comprising the peptides described herein may be used in the manufacturing of a medicament for the treatment of a lung disorder associated with dysregulated VEGF signaling.

In some embodiments, the peptides of the present disclosure, or the pharmaceutical composition comprising such peptides may be administered to a subject in need thereof, in an effective amount to treat a lung disorder associated with dysregulated VEGF signaling. “A therapeutically effective amount” as used herein refers to the amount of peptide required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, therapeutic agents that are compatible with the human immune system, such as peptides comprising regions from humanized antibodies or fully human antibodies, may be used to prolong half-life of the peptide and to prevent the peptide being attacked by the host's immune system. Frequency of administration may be determined and adjusted over the course of therapy, and is generally, but not necessarily, based on treatment and/or suppression and/or amelioration and/or delay of a disorder. Alternatively, sustained continuous release formulations of a peptide may be appropriate. Various formulations and devices for achieving sustained release are known in the art.

The peptides, or the pharmaceutical composition comprising the peptides may be administered repeatedly to a subject (e.g., 2, 3, 4, 5, 6, 7, 8, 9, 10 times or more). In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the peptide used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the peptide (such as the half-life of the peptide, and other considerations well known in the art).

For the purpose of the present disclosure, the appropriate dosage of the peptides as described herein will depend on the specific agent (or compositions thereof) employed, the formulation and route of administration, the type and severity of the disorder, whether the peptide is administered for preventive or therapeutic purposes, previous therapy, the subject's clinical history and response to the antagonist, and the discretion of the attending physician. Typically the clinician will administer a peptide until a dosage is reached that achieves the desired result. Administration of one or more peptides can be continuous or intermittent, depending, for example, upon the recipient's physiological condition, whether the purpose of the administration is therapeutic or prophylactic, and other factors known to skilled practitioners. The administration of a peptide may be essentially continuous over a preselected period of time or may be in a series of spaced dose, e.g., either before, during, or after developing a disorder.

In some embodiments, the subject has a lung disorder associated with dysregulated VEGF signaling. A “lung disorder associated with dysregulated VEGF signaling” is a lung disorder, where the subject having or is at risk of having the lung disorder exhibits any one of the following: (i) secretion of proteases by alveolar neutrophils and macrophages; (ii) decreased level of VEGF in lung cell(s) and/or the lung cell's environment; (iii) decreased VEGF signaling in lung cell(s), and (iv) increased lung endothelial and epithelial cell apoptosis, compared to a healthy control. In some embodiments, the lung disorder is selected from the group consisting of: pulmonary hypertension (PH), neonatal respiratory distress syndrome (RDS), interstitial lung disease associated with systemic sclerosis, acute respiratory distress syndrome (ARDS), chronic obstructive pulmonary disease (COPD), emphysema and bronchopulmonary dysplasia (BPD).

Pulmonary hypertension is a lung disorder characterized by blood pressure in the pulmonary artery that is far above normal levels. Symptoms include shortness of breath, chest pain particularly during physical activity, weakness, fatigue, fainting, light headedness particularly during exercise, dizziness, abnormal heart sounds and murmurs, engorgement of the jugular vein, retention of fluid in the abdomen, legs and ankles, and bluish coloring in the nail bed.

Neonatal respiratory distress syndrome (RDS), is a syndrome in premature infants caused by developmental insufficiency of pulmonary surfactant production and structural immaturity in the lungs. It can also be a consequence of neonatal infection. It can also result from a genetic defect with the production of surfactant associated proteins. RDS affects about 1% of newborn infants and is the leading cause of death in preterm infants.

Interstitial lung disease (ILD) often occurs as a complication of systemic sclerosis (SSc). Lung biopsies of SSc-ILD patients reveal evidence of endothelial and epithelial injury with interstitial edema. Endothelial cell injury results in thrombin production and release of endothelin-1 (ET-1) with elevated levels of thrombin detected in bronchoalveolar lavage (BAL) fluid of SSc patients compared to healthy controls.

Acute respiratory distress syndrome (ARDS), also known as respiratory distress syndrome, or adult respiratory distress syndrome is a condition that arises as a result of injury to the lungs or acute illness. The injury to the lung may be a result of ventilation, trauma, burns, and/or aspiration. The acute illness may be infectious pneumonia or sepsis. It is considered a severe form of acute lung injury, and it is often fatal. It is characterized by lung inflammation, impaired gas exchange, and release of inflammatory mediators, hypoxemia, and multiple organ failure. ARDS can also be defined as the ratio of arterial partial oxygen tension (PaO2) as a fraction of inspired oxygen (FiO2) below 200 mmHg in the presence of bilateral infiltrates on the chest x-ray. A PaO2/FiO2 ratio less than 300 mmHg with bilateral infiltrates indicates acute lung injury, which is often a precursor to ARDS. Symptoms of ARDS include shortness of breath, tachypnea, and mental confusion due to low oxygen levels.

Chronic obstructive pulmonary disease (COPD), is a progressive disorder that makes it hard to breathe. Progressive means the disorder gets worse over time. COPD can cause coughing that produces large amounts of a slimy substance called mucus, wheezing, shortness of breath, chest tightness, and other symptoms. Cigarette smoking is the leading cause of COPD. Most people who have COPD smoke or used to smoke. However, up to 25 percent of people with COPD never smoked. Long-term exposure to other lung irritants—such as air pollution, chemical fumes, or dusts also may contribute to COPD. A rare genetic condition called alpha-1 antitrypsin (AAT) deficiency can also cause the disorder.

Bronchopulmonary dysplasia (BPD) is a condition that afflicts neonates who have been given oxygen or have been on ventilators, or neonates born prematurely particularly those born very prematurely (e.g., those born before 32 weeks of gestation). It is also referred to as neonatal chronic lung discasc. Causes of BPD include mechanical injury for example as a result of ventilation, oxygen toxicity for example as a result of oxygen therapy, and infection. The disorder may progress from non-inflammatory to inflammatory with time. Symptoms include bluish skin, chronic cough, rapid breathing, and shortness of breath. Subjects having BPD are more susceptible to infections such as respiratory syncytial virus infection. Subjects having BPD may develop pulmonary hypertension.

In some embodiments, the lung disorder is emphysema. Emphysema is a chronic progressive pulmonary disorder characterized by gradual thinning, enlargement and destruction of alveoli leading to impaired oxygenation and retention of carbon dioxide that severely threatens human health worldwide. There is currently no effective drug therapy to prevent emphysema progression or restore lung tissue to health.

In some embodiments, the lung disorder is associated with cigarette smoke. A “lung disorder associated with cigarette smoke” refers to a lung disorder that develops after lung cells are exposed to cigarette smoke or any toxic substances contained in cigarette smoke. Exemplary lung disorders associated with cigarette smoke include emphysema, COPD, and idiopathic pulmonary fibrosis (IPF).

In some embodiments, the lung disorder is caused by bacterial lipopolysaccharides (LPS). It is to be understood that the methods described herein is effective in treating lung injury caused by LPS, but does not treat the infection, i.e., the peptide is not anti-microbial. Further, the peptides used in the methods described herein treats the lung disorder caused by LPS via reducing lung cell apoptosis. A second agent (e.g., antibiotics) may be used in connection with the peptide described herein for the treatment of the infection.

In some embodiments, the lung disorder is associated with acute or chronic lung injury. A “lung disorder associated with chronic lung injury” refers to injury caused to lung cells by a chronic lung disorder, causing impaired lung function or disability. In some embodiments, a subject having a lung disorder associated with chronic lung injury may require administration of oxygen intermittently or continuously. A “lung disorder associated with acute lung injury” refers to a condition in which lung function is impaired or lost due to the acute onset of failure of the lung to function, e.g., to oxygenate the blood. Acute lung injury have various causes such as trauma or infection.

In some embodiments, the methods described herein further comprises administering one or more second agents to the subject in need thereof, to treat any of the aforementioned lung disorders. In some embodiments, the second agent treats the symptoms of the lung disorder but does not regulate VEGF signaling, as does the peptide used in the methods described herein. A second agent may be any agent that can be used in the prevention, treatment and/or management of a lung disorder such as those discussed herein. These include but are not limited to surfactants, inhaled nitric oxide, almitrine bismesylate, immunomodulators, and antioxidants. Examples of immunomodulators include steroids and corticosteroids such as but not limited to methylprednisolone. Examples of antioxidants include but are not limited to superoxide dismutase.

Certain agents used in the treatment or management of certain lung disorders including but not limited to pulmonary hypertension include oxygen, anticoagulants such as warfarin (Coumadin); diuretics such as furosemide (Lasix®) or spironalactone (Aldactone®); calcium channel blockers; potassium such as K-dur®; inotropic agents such as digoxin; vasodilators such as nifedipine (Procardia®) or diltiazem (Cardizem®); endothelin receptor antagonists such as bosentan (Tracleer®) and ambrisentan (Letairis®): prostacyclin analogues such as epoprostenol (Flolan®), treprostinil sodium (Remodulin®, Tyvaso®), and iloprost (Ventavis®); and PDE-5 inhibitors such as sildenafil (Revatio®) and tadalafil (Adcirca®).

The peptides may be administered with pulmonary surfactants. A pulmonary surfactant is a lipoprotein mixture useful in keeping lung airways open (e.g., by preventing adhesion of alveolar walls to each other). Pulmonary surfactants may be comprised of phospholipids such as dipalmitoylphosphatidylcholine (DPPC), phosphotidylcholine (PC), phosphotidylglycerol (PG); cholesterol; and proteins such as SP-A, B, C and D. Pulmonary surfactants may be derived from naturally occurring sources such as bovine or porcine lung tissue. Examples include Alveofact™ (from cow lung lavage). Curosurf™ (from minced pig lung), Infasurf™ (from calf lung lavage), and Survanta™ (from minced cow lung, with additional components including DPPC, palmitic acid, and tripalmitin). Pulmonary surfactants may also be synthetic. Examples include Exosurf™ (comprised of DPPC with hexadecanol and tyloxapol), Pumactant™ or Artificial Lung Expanding Compound (ALEC) (comprised of DPPC and PG), KL-4 (comprised of DPPC, palmitoyl-oleoyl phosphatidylglyercol, palmitic acid, and synthetic peptide that mimics SP-B), Venticute™ (comprised of DPPC, PG, palmitic acid, and recombinant SP-C). Pulmonary surfactants may be obtained from commercial suppliers.

A “subject in need thereof” refers to a subject who has or is at risk of having dysregulated VEGF signaling in the lungs and/or an associated disorder. In some embodiments, the subject is a mammal. In some embodiments, the subject is a non-human primate. In some embodiments, the subject is human. In some embodiments, the subject is an infant, e.g., a human infant. In some embodiments, the mammal is a rodent, such as a mouse or a rat.

As used herein, the term “treating” refers to the application or administration of a peptide or composition including the peptide to a subject in need thereof. “A subject in need thereof”, refers to an individual who has a disorder, a symptom of the disorder, or a predisposition toward the disorder, with the purpose to cure, heal, alleviate, relieve, alter, remedy, ameliorate, alleviate, improve, or affect the disorder, the symptom of the disorder, or the predisposition toward the disorder.

Alleviating a disorder includes delaying the development or progression of the disorder, or reducing disorder severity. Alleviating the disorder does not necessarily require curative results. As used therein, “delaying” the development of a disorder means to defer, hinder, slow, retard, stabilize, and/or postpone progression of the disorder. This delay can be of varying lengths of time, depending on the history of the disorder and/or individuals being treated. A method that “delays” or alleviates the development of a disorder, or delays the onset of the disorder, is a method that reduces probability of developing one or more symptoms of the disorder in a given time frame and/or reduces extent of the symptoms in a given time frame, when compared to not using the method. Such comparisons are typically based on clinical studies, using a number of subjects sufficient to give a statistically significant result.

“Development” or “progression” of a disorder means initial manifestations and/or ensuing progression of the disorder. Development of the disorder can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disorder includes initial onset and/or recurrence.

Conventional methods, known to those of ordinary skill in the art of medicine, can be used to administer the peptide or pharmaceutical composition to the subject, depending upon the type of disorder to be treated or the site of the disorder. The peptide or composition comprising the peptide can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the peptide or the pharmaceutical composition comprising the peptide is administered systemically. In some embodiments, the peptide or the pharmaceutical composition comprising the peptide is administered directly to the lungs, e.g., via inhalation or instillation. In some embodiments, instillation may be used to deliver the peptide or the pharmaceutic composition comprising the peptide to a subject who is intubated (e.g., on a respirator in the hospital) or who has a tracheotomy. In some embodiments, the peptide or the pharmaceutical composition comprising the peptide can be administered to the subject via injectable depot routes of administration such as using 1-, 3-, or 6-month depot injectable or biodegradable materials and methods.

In some embodiments, the peptide or the pharmaceutical composition comprising the peptide is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the peptide of the disclosure does not absorb are used.

In other embodiments, the peptide or the pharmaceutical composition comprising the peptide is delivered in a controlled release system. In some embodiments, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a pharmaceutical composition adapted for intravenous or subcutaneous administration to a subject, e.g., a human being. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. The peptides of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4.920,016; and 4,921,757.

The pharmaceutical compositions of the present disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a peptide of the disclosure in lyophilized form and (b) a second container containing a pharmaceutically acceptable diluent (e.g., sterile water) for injection. The pharmaceutically acceptable diluent can be used for reconstitution or dilution of the lyophilized peptide of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the disorders described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disorder described herein and may have a sterile access port. For example, the container may be an intravenous solution bag or a vial having a stopper pierceable by a hypodermic injection needle. The active agent in the composition is an isolated peptide of the disclosure. In some embodiments, the label on or associated with the container indicates that the composition is used for treating the disorder of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: PR1P, a Novel VEGF Stabilizing Peptide, Mitigates Cigarette Smoke In Vitro and LPS Induced Lung Cell Apoptosis In Vivo

Emphysema is a chronic, progressive and fatal pulmonary disorder that lacks an effective drug therapy to prevent its progression or restore lung tissue to health (1). Emphysema is characterized by gradual thinning, enlargement and destruction of alveoli leading to impaired oxygenation and retention of carbon dioxide that severely threatens human health (2). It is projected to become the third leading cause of adult mortality worldwide by 2030 (2). Although the mechanisms are incompletely understood, evidence suggests that disease progression is due in part to 1) chronic lung inflammation, 2) increased secretion of proteases by alveolar neutrophils and macrophages within the lung parenchyma (3-5), 3) dysregulation within the lungs of the survival and angiogenic Vascular Endothelial Growth Factor (VEGF) and its receptor VEGFR-2 (5-8), and 4) increased lung endothelial and epithelial cell apoptosis (9, 10). Because VEGF is curiously expressed at 500-fold higher concentration in the lungs compared to serum (11, 12), it is thought to play a significant role in maintaining lung tissue homeostasis. As it is directly or indirectly implicated in mediating each of the aforementioned disease mechanisms responsible for emphysema, recent interest in emphysema research has focused on developing pharmaceuticals to target and manipulate VEGF signaling.

VEGF₁₆₅ (a 165 amino acid isoform of VEGF-A, henceforth referred to as VEGF) is a ubiquitous angiogenic and survival factor that binds two tyrosine kinase receptors, VEGF receptor 1 (VEGFR1) and VEGF receptor 2 (VEGFR-2), as well as neuropilin-1 (NRP-1) (13). VEGFR1 and VEGFR-2 are each expressed to varying degrees on lung endothelial, epithelial and alveolar macrophages (14, 15). VEGF stimulates endothelial and epithelial cell proliferation, and blood vessel formation, and enhances cell survival in part through inhibition of signaling pathways that lead to cell apoptosis (16-18). There is compelling evidence to suggest that VEGF dysregulation contributes to the development of emphysema (19). Firstly, pharmacological inhibition or molecular modifications of VEGF or of VEGFR-2 result in an emphysema phenotype in rodents (20). Secondly, studies in humans with emphysema and in animal emphysema models show a strong correlation between emphysema disease progression and reduced levels of both VEGF and VEGFR-2 and increased lung cell apoptosis (21, 22). Finally, in vitro work using emphysema models, including with cigarette smoke and lipopolysaccharide (LPS), suggests that VEGF modulates lung endothelial cell apoptosis and cell survival in part through signaling through VEGF receptors and downstream activation of phosphoinositide-3 kinase (PI3K)/Akt (23-26). Whether dysregulation of VEGF leads to emphysema or is a byproduct of this disorder is not entirely clear.

Recent studies suggest that VEGF signaling may be mediated in part by the local microenvironment. Local tissue VEGF concentrations dramatically rise or fall during acute tissue or organ injury (i.e. in wounds (27), myocardial infarction (28), peripheral vascular occlusive disease (29), acute respiratory distress syndrome (ARDS) (30)) and then return to baseline following recovery (31). One hypothesis is that VEGF signaling is mediated locally at these sites of tissue injury during acute or chronic illness by dynamic switches in protein degradation kinetics mediated by inflammatory cell signaling (32). For example, products of inflammatory cells such as the proteases plasmin and elastase, which are increased in human emphysema as well as in animal models of emphysema, cleave active VEGF dimers into smaller VEGF degradation products with altered receptor binding and signaling properties thereby reducing VEGF activity (33, 34). A clear understanding of how inflammation induced alterations in VEGF processing modulate lung cell and tissue health in disease states such as emphysema remains elusive. It was hypothesized that a pharmaceutical that targets and stabilizes endogenous VEGF could be used in emphysema to enhance endogenous VEGF signaling and restore lung cell and tissue health.

To begin to test this hypothesis, the recent discovery that the pentaspan transmembrane glycoprotein prominin-1 bound VEGF and potentiated its anti-apoptotic and pro-angiogenic activities in vitro and in vivo was employed (35). Based on these findings, a novel 12 amino acid peptide, PR1P, whose sequence was derived from one of the extracellular VEGF binding domains of Prominin-1, was designed (36). It was recently shown that PR1P also bound VEGF and increased VEGF binding to VEGF receptors VEGFR2 and Neuropilin-1, and to endothelial cells in vitro (36). Importantly, PR1P increased VEGF dependent angiogenesis in multiple murine angiogenesis models in vivo, including in a corneal micropocket assay, in choroidal neovascularization, wound-healing models, and augmented reperfusion in a murine hind-limb ischemia model (36). Herein it is shown that PR1P upregulated VEGF signaling as evidenced by its augmentation of VEGFR-2 and AKT phosphorylation in human epithelial lung cells in vitro, and in murine lung cells in vivo following PR1P inhalation by spontaneously breathing animals. PR1P also reduced cigarette smoke extract (CSE) induced lung epithelial cell apoptosis in an in-vitro emphysema model, and inhaled LPS induced lung epithelial cell apoptosis in-vivo. 3D computer simulations and dot blot binding studies supported the hypothesis that PR1P bound VEGF on or near its heparin binding domain where proteolytic enzymes plasmin and elastase bind, which suggests that PR1P may augment VEGF signaling by protecting it from proteolytic degradation. This theory was tested and it was found that PR1P limited VEGF degradation in vitro by both plasmin and elastase. Together these studies suggest that PR1P may augment endogenous VEGF signaling by interfering with its degradation by proteases present within the lungs during acute and chronic inflammation. Moreover, the data described herein points to a potential critical role for PR1P in reducing apoptosis of epithelial and endothelial cells in emphysema, and in other lung or systemic disorders characterized by VEGF signaling dysregulation.

### Results

### PR1P Stimulated VEGF Signaling In Vitro and In Vivo

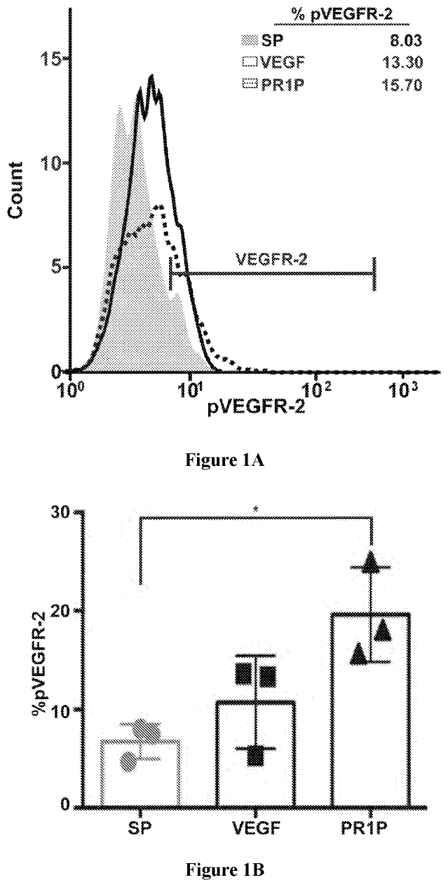

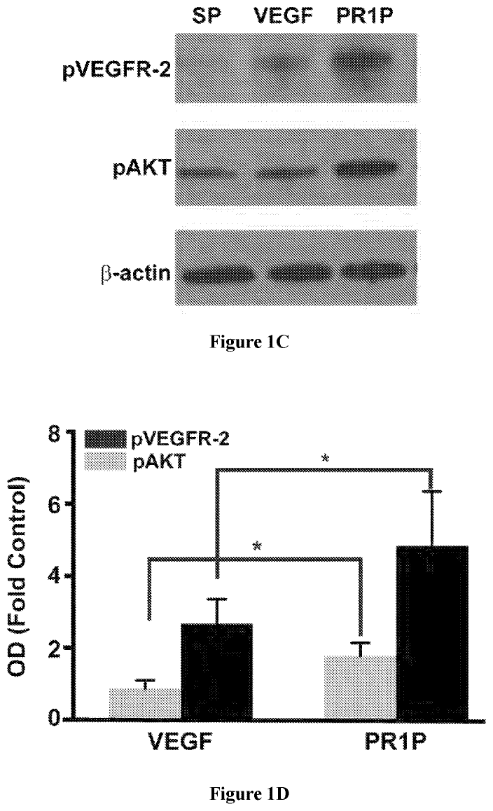

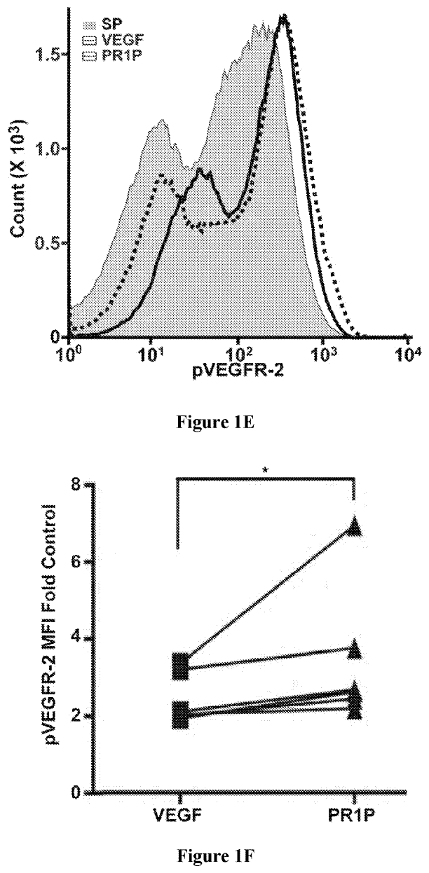

VEGF mediates cell survival and apoptosis pathways via binding to and activation of VEGFR-2 and signaling through the PI3K/AKT pathway (13). It was recently shown that PR1P, a novel 12 amino acid peptide, was designed that was derived from an extracellular VEGF binding domain of Prominin-1 (35), bound VEGF, increased VEGF binding to VEGF receptors VEGFR2 and Neuropilin-1 and to endothelial cells in vitro (36), and increased VEGF dependent angiogenesis in multiple murine angiogenesis models in vivo (36). To evaluate the effect of PR1P on VEGF signaling in the lung, both FACS and Western blotting were used to measure the levels of phosphorylated VEGFR-2 and AKT in the human lung respiratory epithelial cell line BEAS-2B in response to treatment with PR1P, scrambled peptide (SP), or VEGF. As shown in FIGS. 1A-1D, incubation of BEAS-2B cells with PR1P significantly increased pVEGFR-2 (FIGS. 1A-1D) and AKT phosphorylation (FIGS. 1C and 1D) compared to incubation with SP or VEGF alone. To determine whether PR1P also effectively modulated VEGF signaling in the lungs of animals (i.e. whether PR1P was bioactive in-vivo) live mice were treated with nebulized VEGF, SP or PR1P (20 min) and performed FACS analysis on lung cells harvested from treated animals 30 minutes after the end of inhalation. FIGS. 1E and 1F show that inhaled PR1P increased lung cell VEGFR-2 phosphorylation compared to SP or to VEGF alone. Together, this data strongly suggests that PR1P activates VEGF signaling pathway in-vitro and in-vivo, which led to further evaluation whether it will improve VEGF activity as a survival factor

### PR1P Decreased Cigarette Smoke Induced Apoptosis In-Vitro and LPS Induced Apoptosis In-Vivo

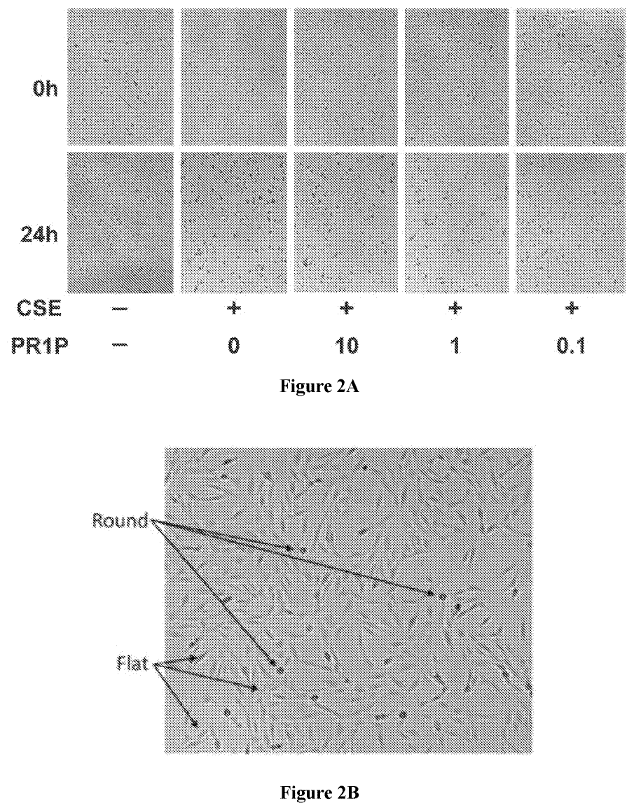

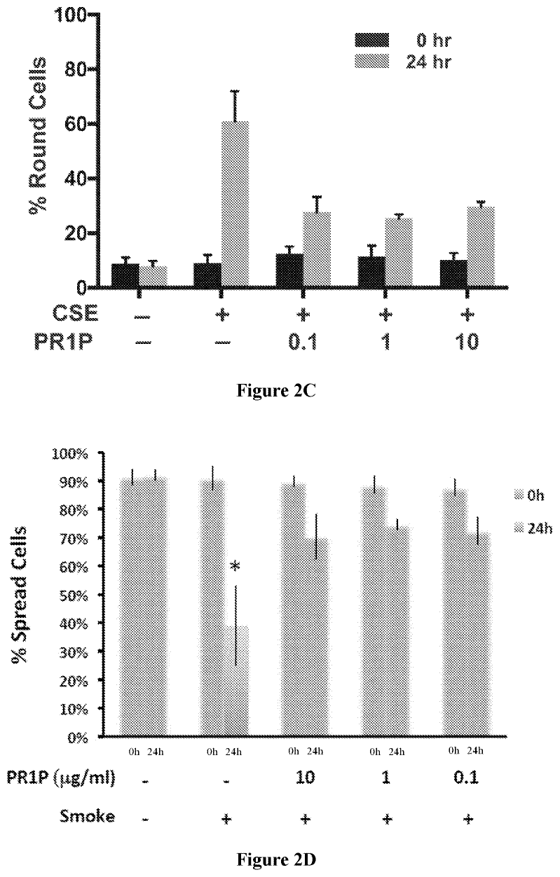

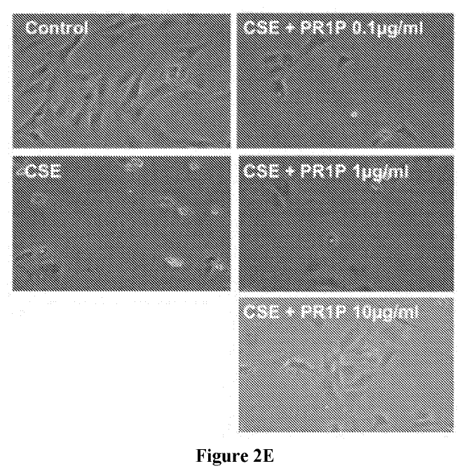

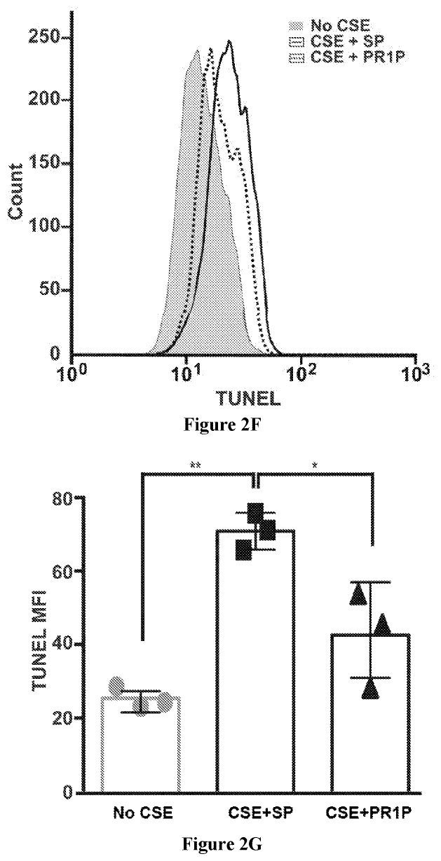

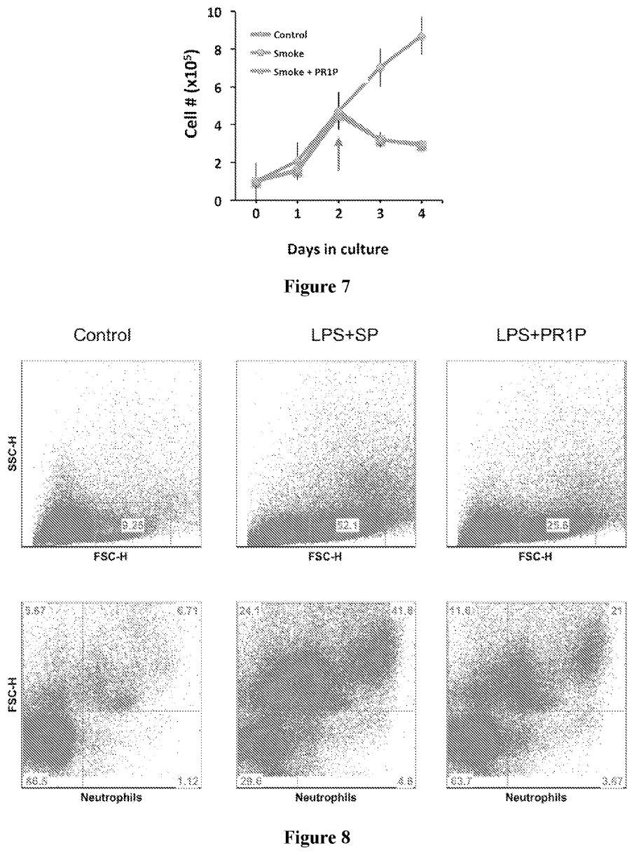

To examine whether PR1P could be used to improve cell survival following toxic injury, it was evaluated whether PR1P rescued lung epithelial cells in vitro from cigarette smoke extract (CSE) induced apoptosis (25). To begin these studies, BEAS-2B cells were incubated with increasing concentrations of CSE for 48 hours in order to select an optimal CSE concentration for the studies that would prevent cell proliferation, but also have minimal effect on cell death (see Methods). FIG. 7 shows that 3% CSE exposed stock media (which was used for all subsequent studies) had a negligible effect on cell death, but did arrest cell proliferation (FIG. 7). Interestingly, despite the ability to augment VEGF signaling (see FIGS. 1A-1F). PR1P neither prevented the marginal degree of cell death nor enabled ongoing cell proliferation (FIG. 7). However, microscopic examination of fixed cells that were similarly treated to those described above revealed that CSE induced a marked increase in the number of cells at 24 hours post CSE exposure that had a round cell morphology in comparison to a flat (well spread) cell morphology that was significantly reduced in the presence of PR1P (FIGS. 2A-2C). Staining of these cells with TUNEL to identify apoptosis revealed that the round cells stained with TUNEL, whereas “flat” cells did not, suggesting a strong correlation between the round cell morphology and apoptosis following exposure to CSE (FIG. 2E). To confirm this and to quantify the effect of PR1P on CSE induced apoptosis, these same cells were treated in parallel experiments and then prepared for FACS analysis. It was confirmed that CSE induced BEAS-2B cell apoptosis was significantly mitigated by treatment with PR1P (FIGS. 2F-2G).

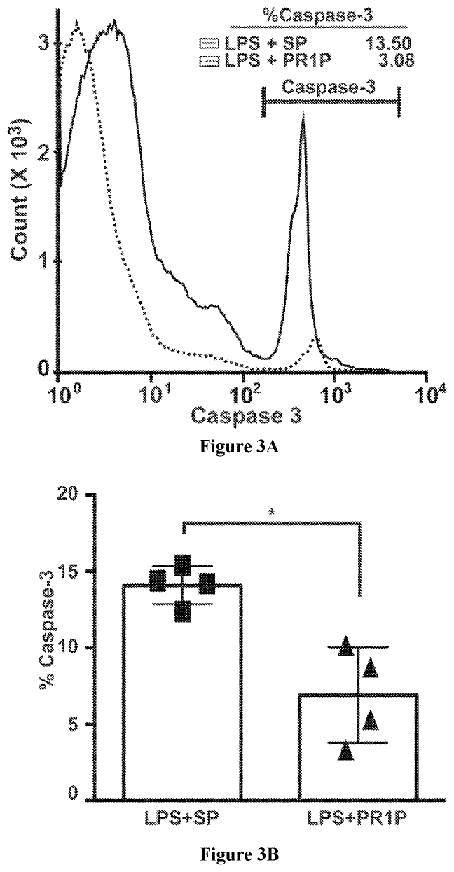

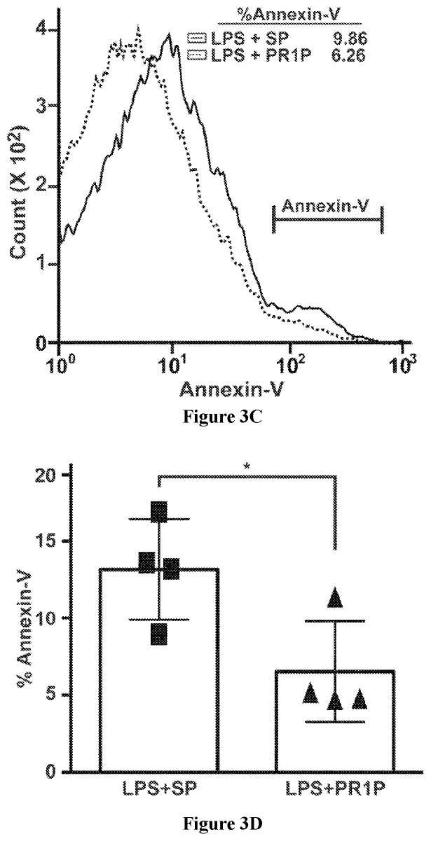

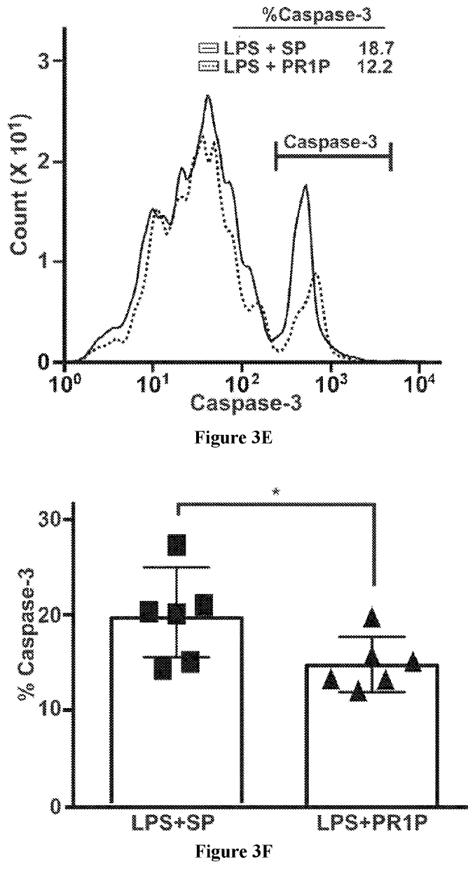

Considering that it is shown herein that PR1P is bioactive when inhaled into living mice (see FIGS. 1E-1F), it was next sought to determine whether PR1P protects lung cells from toxin induced apoptosis in-vivo. Cigarette tobacco contains large amounts of substances of microbiological origin including lipopolysaccharide (LPS, (37)), which is known to induce lung cell apoptosis in murine lung injury models in-vivo (38). Therefore, whether PR1P mitigates apoptosis of lung cells, and specifically lung epithelial cells, in mice treated with nebulized LPS was explored. As shown in FIGS. 3A-3D, inhalation by live mice of PR1P after LPS inhalation significantly reduced LPS-induced apoptosis of all lung cells (FIGS. 3A-3D), and specifically of lung epithelial cells (FIGS. 3E-3F). Taken together, these results suggest that PR1P protects lung cells in-vitro and in-vivo from toxins that induce lung cell apoptosis.

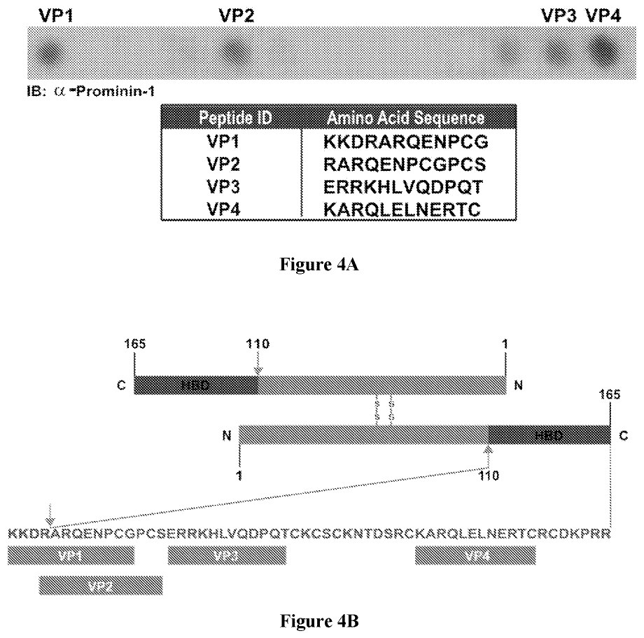

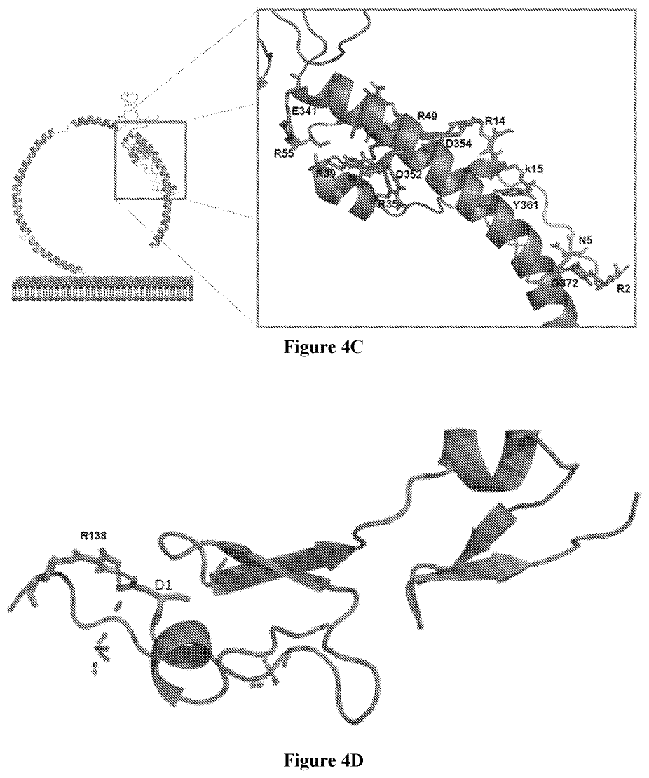

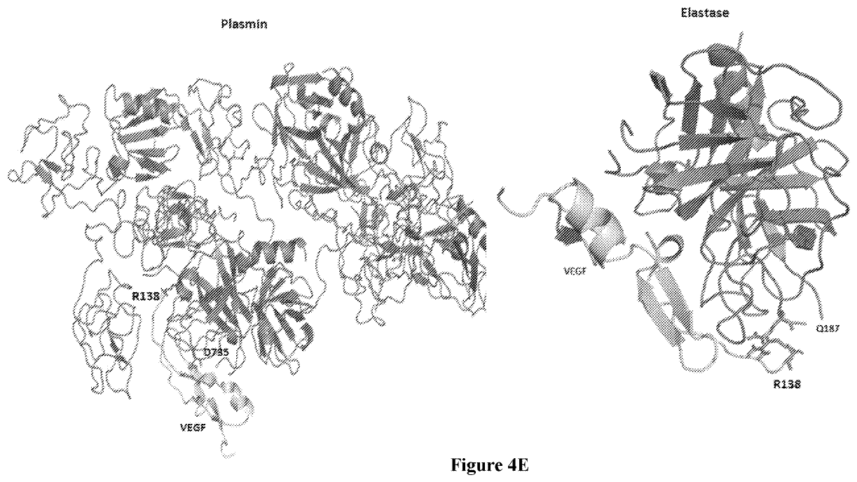

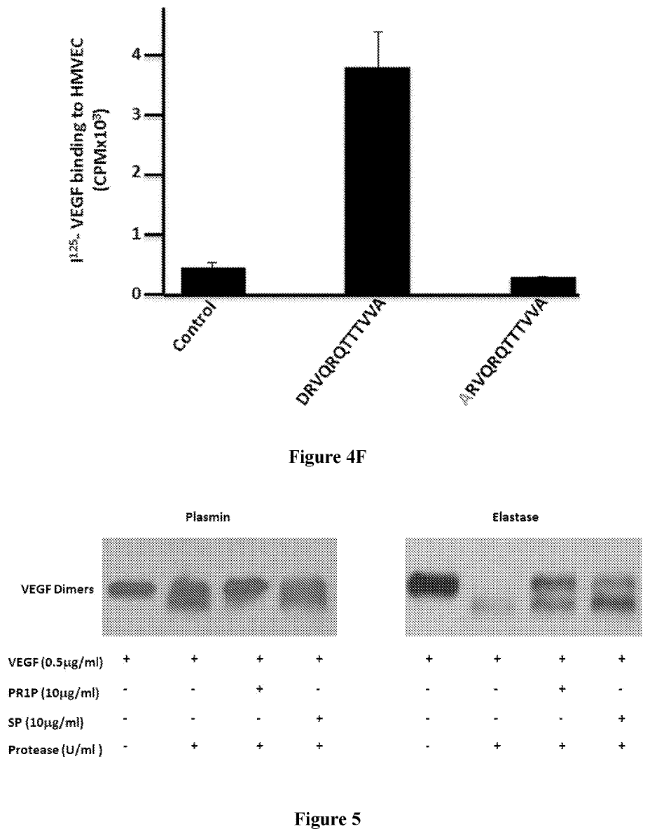

It has been shown in the past that Prominin-1 bound VEGF (35), as did multiple 12 amino acid oligomers comprised of different extracellular regions of Prominin-1, including PR1P (36). It was hypothesized that a broader understanding of how Prominin-1 and PR1P bind VEGF would provide insight into the PR1P mechanism of action. To determine the sequences within VEGF that permit its' binding by prominin-1,12-amino acid long peptides containing overlapping sequences from VEGF were designed and manufactured. Then the peptides' affinity for binding to Prominin-1 was tested using a cellulose peptide binding array. Here, membranes with transferred short peptides containing sequences from VEGF were incubated with Prominin-1 and blotted with anti-Prominin-1 antibody (FIG. 4A). Blots revealed that the 4 VEGF derived peptides (blot and sequences shown in FIG. 4A) amongst 179 tested that displayed the greatest binding of Prominin-1 were each derived from the heparin binding domain (HBD) of VEGF (FIG. 4B).

To provide further insight into how Prominin-1 may bind VEGF near or within the VEGF HBD, 3D computer modeling was used to determine high probability molecular binding interactions between the extracellular VEGF binding domain of Prominin-1 that contains PR1P and the HBD-containing sequence of VEGF. FIG. 4C shows the computer simulation of the prominin-1 extracellular domain binding to the VEGF HBD fragment and Tables 1 and 2 list the frequencies of hydrogen bond formations between these two molecules during docking experiments. The results herein indicate that residues E341, R246, D352, Q372 and D354 in the Prominin-1 fragment and residues R39, R35 and R49 and K30 in the VEGF HBD fragment are highly likely to interact during association. Interestingly, residues Q372, Q374 and T375 in the prominin-1 fragment, which show moderately high frequency of interaction with the VEGF-HBD, are contained within PR1P. To more clearly define how the PR1P peptide alone might interact with VEGF, next PR1P binding was simulated with the same VEGF HBD containing fragment. 3D computation modeling and docking experiments showed that aspartic acid in position 1 in PR1P (D1 in FIG. 4D) was highly likely to form a hydrogen bond with R138 of the VEGF HBD containing fragment (Tables 3 and 4). Note that R138 on VEGF serves as a cleavage site for the proteases plasmin and elastase (34, 39) (and see plasmin cleavage site (green arrow) within the HBD (aa 110) in FIG. 4B). To determine the likelihood that PR1P might interfere with these proteases from binding to VEGF, docking experiments were simulated between clastase or plasmin and the VEGF HBD in the absence or presence of PR1P. As shown in FIG. 4E, the simulation results herein suggest that PR1P binding to the VEGF HBD at R138 competitively inhibited binding of both plasmin and elastase to this same residue and sterically altered the three dimensional conformation of the proteolytic enzymes during docking. To confirm the importance of aspartic acid at position 1 (D1) within the PR1P sequence, a modified PR1P peptide that contained an alanine (A) in the first position instead of aspartic acid (D) was generated and the ability of this modified peptide to that of PR1P to enhance radioactive VEGF binding to endothelial cells in vitro was compared as done in the past (36). As shown in FIG. 4F, substitution of aspartic acid at position 1 in PR1P (corresponding to D1 in FIG. 4B) with alanine reduced the effect of the peptide on VEGF binding to endothelial cells. This data suggests that the aspartic acid in position 1 of PR1P is critical in enabling PR1P binding to VEGF and in particular at or on the HBD. Because the activity and concentration of proteases such as plasmin and elastase are increased during acute and chronic inflammation seen in emphysema and other lung disorders, it was hypothesized that PR1P may protect VEGF from proteolysis by competing with proteases for binding sites within the VEGF HBD.

Table 1 shows the frequency of H-bond formation in 3D simulation and docking experiments for Prominin-1-EC domain and the HBD containing fragment of VEGF. There were a total of 200 models per each docking experiments. The frequency of residues in Prominin-1 and VEGF (HBD) to form H-bonds was ranked among these models respectively in Table 1. From this modeling, it was found that R224, D295, D229, T292 and R246 in Prominin-1 and R2, K15, E4, R46, R35 AND R49 in VEGF (HBD) are critical residues to form the complex. The crystal structure of human VEGF (HBD) was taken from PDB database (PDB ID: 1KMX). Simulation for Prominin-1 (N206-T296) was performed using SWISS-MODEL and the docking experiments of Prominin-1 and VEGF (HBD) were performed using the ClusPro 2.0 program.

TABLE 1
Interaction residues between prominin-1_II and VEGF (HBD).

| Interaction residues of Prominin-1 | | Interaction residues of VEGF | |
|---|---|---|---|
| to form H-bonds | | (HBD) to form H-bonds | |
| Predicted | | Predicted | |
| residues in | Frequency to | residues in | Frequency to |
| Prominin-1 | form H-bonds | VEGF (HBD) | form H-bonds |
| R224 | 30 | R2 | 34 |
| D295 | 16 | K15 | 24 |
| D229 | 15 | E4 | 24 |
| T292 | 14 | Q20 | 20 |
| R246 | 13 | A1 | 16 |
| N231 | 13 | R46 | 14 |
| D254 | 12 | Q3 | 14 |
| D243 | 11 | D21 | 13 |
| S235 | 10 | K26 | 11 |
| E255 | 10 | R35 | 11 |
| Q286 | 9 | R13 | 10 |
| K257 | 8 | N5 | 10 |
| S293 | 8 | S28 | 10 |
| S289 | 8 | S34 | 9 |
| N274 | 8 | R49 | 8 |
| L245 | 7 | C10 | 8 |
| N248 | 7 | E12 | 7 |
| N248 | 7 | E45 | 7 |
| N234 | 7 | K30 | 7 |
| T225 | 6 | Q23 | 6 |
| L291 | 6 | R14 | 6 |
| Q218 | 6 | C7 | 6 |
| S284 | 5 | T32 | 6 |
| S297 | 5 | E12 | 5 |
| Y214 | 5 | H16 | 5 |
| L230 | 5 | R55 | 5 |
| T285 | 5 | E42 | 4 |
| K223 | 5 | S11 | 4 |
| G239 | 4 | D51 | 3 |
| V294 | 4 | P6 | 3 |
| S275 | 4 | K52 | 2 |
| L242 | 4 | R3 | 1 |
| N124 | 4 | Q4 | 1 |
| L270 | 4 | | |
| L287 | 3 | | |
| Q283 | 3 | | |
| N272 | 3 | | |
| K257 | 3 | | |
| K267 | 3 | | |
| T222 | 3 | | |
| N234 | 3 | | |
| S301 | 3 | | |
| K278 | 3 | | |
| S232 | 2 | | |
| S258 | 2 | | |
| E271 | 2 | | |
| E210 | 1 | | |
| R299 | 1 | | |

Table 2 shows the frequency of H-bond formation in 3D simulation and docking experiments for Prominin-1-EC domain and the HBD containing fragment of VEGF. There were a total of 200 models per each docking experiments. The frequency of residues in Prominin-1 and VEGF (HBD) to form H-bonds was ranked among these models respectively in Table 2. From the modeling, it was found that E341, R246, D352, Q372 and D354 in Prominin-1 and R39, R35 and R49 AND K30 in VEGF (HBD) are critical residues to form the complex. Very interestingly, Q372, Q374 and T375 residues were present in the PIP synthetic peptide. The crystal structure of human VEGF (HBD) was taken from PDB database (PDB ID: 1KMX). Simulation for Prominin-1 (K192-Q398) was performed using SWISS-MODEL and the docking experiments of Prominin-1 and VEGF (HBD) were performed using ClusPro 2.0 program.

TABLE 2
Interaction residues between prominin-1 and VEGF (HBD).

| Interaction residues of Prominin-1 | | Interaction residues of VEGF | |
|---|---|---|---|
| to form H-bonds | | (HBD) to form H-bonds | |
| Predicted | | Predicted | |
| residues in | Frequency to | residues in | Frequency to |
| Prominin-1 | form H-bonds | VEGF (HBD) | form H-bonds |
| E341 | 11 | R39 | 20 |
| R246 | 11 | R35 | 19 |
| D352 | 10 | R2 | 15 |
| Q372 | 9 | R49 | 14 |
| D354 | 9 | K30 | 13 |
| N346 | 7 | R14 | 12 |
| T266 | 6 | E4 | 11 |
| Q334 | 6 | R46 | 10 |
| Q320 | 6 | R55 | 9 |
| Q374 | 5 | K26 | 7 |
| D343 | 5 | D21 | 7 |
| N303 | 5 | R13 | 6 |
| L335 | 4 | K52 | 6 |
| Q359 | 4 | N5 | 6 |
| E255 | 4 | A1 | 5 |
| N365 | 4 | Q3 | 5 |
| D339 | 4 | H16 | 5 |
| S363 | 4 | Q40 | 4 |
| L309 | 4 | E12 | 4 |
| L335 | 4 | Q23 | 3 |
| S328 | 3 | C7 | 3 |
| N329 | 3 | D51 | 2 |
| S322 | 3 | E45 | 2 |
| N177 | 3 | T47 | 2 |
| S318 | 3 | P6 | 2 |
| E331 | 3 | P9 | 2 |
| L364 | 3 | C10 | 2 |
| K295 | 3 | R45 | 1 |
| D369 | 3 | N31 | 1 |
| E265 | 3 | N44 | 1 |
| N248 | 3 | A38 | 1 |
| V357 | 3 | P22 | 1 |
| L302 | 3 | S11 | 1 |
| E314 | 2 | R48 | 1 |
| T375 | 2 | S28 | 1 |
| R384 | 2 | C50 | 1 |
| S301 | 2 | S34 | 1 |
| D304 | 2 | K37 | 1 |
| R350 | 2 | | |
| S284 | 2 | | |
| L356 | 2 | | |
| Y219 | 2 | | |
| K257 | 2 | | |
| D254 | 2 | | |
| N220 | 2 | | |
| R299 | 2 | | |

Table 3 shows the frequency of H-bond formation in 3D simulation and docking experiments for PR1P and the HBD containing fragment of VEGF. There were a total of 200 models per each docking experiments. The frequency of residues in Plasmin and VEGF_A137-R191 to form H-bonds was ranked among these models respectively in Table 3. From the modeling, it was found that E459, D735 and R561 in Plasmin and A1, R2, Q3, E4,R13, and K 15 in VEGF arc critical residues to form the complex. Very interestingly, A1, R2, Q3 and E4 are all located in the potential plasmin cleavage site of VEGF. The crystal structure of human VEGF (PDB ID: 1KMX) and Plasmin (PDB ID: 4DUR) were taken from PDB database and the docking experiments of Elastase and VEGF (HBD) were performed using the ClusPro 2.0 program.

TABLE 3
Interaction residues between Plasmin and VEGF (HBD).

| Interaction residues of Elastase | | Interaction residues of | |
|---|---|---|---|
| to form H-bonds | | VEGF_A137-R191 to form H-bonds | |
| Predicted | | Predicted | |
| residues in | Frequency to | residues in | Frequency to |
| Plasmin | form H-bonds | VEGF (HBD) | form H-bonds |
| E459 | 13 | R13 (R149) | 35 |
| D735 | 9 | R2 (R138) | 32 |
| R561 | 8 | K15 (K151) | 31 |
| T691 | 7 | E4 | 27 |
| R220 | 7 | R35 | 24 |
| R367 | 6 | Q3 | 22 |
| D357 | 6 | A1 | 20 |
| R312 | 6 | R39 | 19 |
| S365 | 6 | Q23 | 17 |
| H629 | 5 | E12 | 16 |
| K661 | 5 | Q20 | 15 |
| K556 | 5 | N5 | 14 |
| S436 | 5 | H16 | 14 |
| S545 | 5 | R49 | 14 |
| N399 | 5 | C7 | 12 |
| F692 | 5 | K30 | 9 |
| Y327 | 4 | R14 | 9 |
| R117 | 4 | R39 | 6 |
| Q631 | 4 | R46 | 6 |
| P544 | 4 | D21 | 4 |
| D362 | 4 | P22 | 4 |
| K433 | 4 | | |
| Q364 | 4 | | |
| S654 | 4 | | |
| E395 | 3 | | |

Table 4 shows the frequency of H-bond formation in 3D simulation and docking experiments for PR1P and the HBD containing fragment of VEGF. There were a total of 200 models per each docking experiments. The frequency of residues in Elastase and VEGF_A137-R191 to form H-bonds was ranked among these models respectively in Table 4. From the modeling, it was found that R36, R146 and G218 in Elastase and R2, R13 and K 15 (this corresponds to the residucs R137, R149 and K151 in full length of VEGF in VEGF) arc critical residues to form the complex. Very interestingly, R138 is located in the potential Elastase cleavage site of VEGF. The crystal structure of human VEGF (PDB ID: 1KMX) and Elastase (PDB ID: 4WVP) were taken from PDB database. The docking experiments of Elastase and VEGF (HBD) were performed using the ClusPro 2.0 program.

TABLE 4
Interaction residues between Elastase and VEGF (HBD).

| | | Interaction residues of | |
|---|---|---|---|
| Interaction residues of Elastase | | VEGF_A137-R191 to | |
| to form H-bonds | | form H-bonds | |
| Predicted | | Predicted | |
| residues in | Frequency to | residues in | Frequency to |
| Elastase | form H-bonds | VEGF (HBD) | form H-bonds |
| R36 | 22 | R13 (R149) | 22 |
| R146 | 17 | R2 (R138) | 20 |
| G218 | 17 | K15 (K151) | 16 |
| N61 | 13 | R35 | 14 |
| Y224 | 13 | Q20 | 14 |
| V216 | 11 | E4 | 14 |
| V97 | 9 | R14 | 12 |
| H57 | 8 | E12 | 11 |
| P96 | 8 | N5 | 10 |
| G219 | 8 | R39 | 9 |
| R63 | 7 | Q23 | 8 |
| Y94 | 7 | R46 | 7 |
| R148 | 7 | A1 | 6 |
| R146 | 7 | Q3 | 6 |
| H40 | 6 | C7 | 6 |
| N98 | 6 | H16 | 6 |
| G145 | 6 | K30 | 6 |
| A60 | 5 | R49 | 6 |
| R65 | 5 | D21 | 5 |
| S214 | 5 | S28 | 5 |
| Q187 | 5 | D51 | 5 |
| C58 | 5 | P6 | 4 |
| L35 | 4 | K26 | 4 |
| Q187 | 4 | E45 | 4 |
| R76 | 3 | T24 | 4 |
| S153 | 3 | S11 | 4 |
| G193 | 3 | G8 | 3 |
| S74 | 2 | D33 | 3 |
| E90 | 2 | K52 | 3 |
| V62 | 2 | T32 | 3 |
| I151 | 2 | C7 | 2 |
| F191 | 2 | P9 | 2 |
| S221 | 2 | C10 | 2 |
| L223 | 2 | P22 | 2 |
| D102 | 2 | K33 | 2 |
| R186 | 2 | S34 | 2 |
| T164 | 2 | D21 | 1 |
| C220 | 2 | C25 | 1 |
| G38 | 1 | N31 | 1 |
| F41 | 1 | N43 | 1 |
| E77 | 1 | P53 | 1 |
| L73 | 1 | E42 | 1 |
| R117 | | N44 | 1 |
| L166 | | | |
| C168 | | | |
| P225 | | | |

### PR1P Protects VEGF from Protease Degradation

To test whether PR1P protects VEGF from protease degradation, VEGF (500 ng/ml) was incubated in vitro with plasmin (1 U/mL) in binding buffer at 37° C. for 2 h in the presence and absence of PR1P or SP (10 μg/mL) and the sizes of resultant VEGF degradation products were evaluated under non-reducing conditions by western blot analysis. Note that plasmin cleaves VEGF at amino acid 110 rendering VEGF fragments with an approximate molecular mass of 45-48 kDa (40). Non-reducing conditions were used for enzyme incubation and Western blotting experiments because this allowed VEGF to remain in its native active disulfide linked dimeric form (40). Interestingly, it was found that PR1P significantly reduced the proportion of plasmin generated VEGF fragments (FIG. 5A). Similarly VEGF (500 ng/mL) was exposed to the protease elastase (1 U/mL) which cleaves VEGF at both amino and carboxy terminal ends (34), in the presence and absence of PR1P (or SP (10 g/mL) and found that PR1P also reduced the proportion of elastase generated VEGF fragments (FIG. 5B). Together, this data strongly supports the notion that PR1P augments VEGF signaling by binding to and stabilizing VEGF by preventing its degradation by proteases.

### Discussion

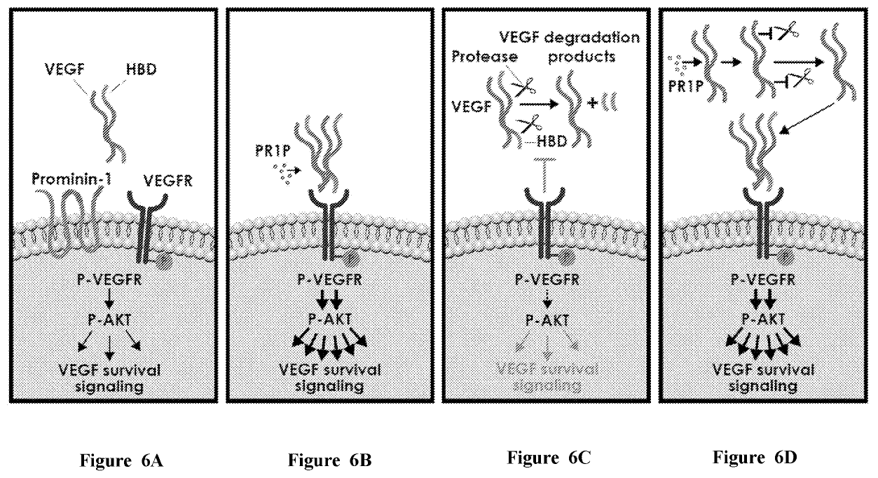

Herein it is shown that PR1P, a novel VEGF binding peptide with angiogenic properties (36), increased lung cell survival by upregulating endogenous VEGF signaling and reducing cigarette smoke and LPS induced apoptosis in-vitro and in-vivo, respectively. Molecular biology studies and computer simulation supported the hypothesis that PR1P binds and stabilizes VEGF from proteolytic degradation, and thus represents a novel approach of stimulating endogenous VEGF signaling with a small VEGF targeting peptide. A schematic summarizing these findings and a hypothetical model of the effect of PR1P on VEGF signaling is presented in FIG. 6. The studies described herein suggest that PR1P could be used to limit apoptosis and improve lung cell and tissue health in patients with emphysema, a debilitating disorder with hallmark features of VEGF dysregulation, and a disorder that has no cure. Using PR1P to target and up-regulate endogenous VEGF in the lungs could also eliminate toxicity associated with systemic VEGF therapy (41).

3D computer simulations of molecular binding interactions between Prominin-1 or PR1P and VEGF predicted that PR1P bound VEGF near the VEGF HBD. These studies, in combination with cellulose peptide arrays showing that Prominin-1 bound VEGF sequences containing the HBD, led to the hypothesis that PR1P binding to VEGF might attenuate VEGF degradation by proteases which also bind VEGF at or near the HBD. This hypothesis was supported by in vitro studies which showed that PR1P limited VEGF degradation by both elastase and plasmin which importantly, are both elevated during chronic inflammation associated with emphysema. VEGF stability and signaling are mediated by local factors in tissue microenvironments that regulate VEGF gene transcription (42, 43), mRNA stabilization (44) and translation (45), and these factors differ dramatically during health and disease (42). Once in its active disulfide linked heterodimeric form (46, 47), VEGF may be modified or degraded by proteases including plasmin and elastase (34, 40, 48, 49) that are elevated in tissues during acute and chronic inflammation (50) and in emphysema specifically due to reduced levels of the protease inhibitor alpha-1 anti-trypsin (51). Chronic unresolving ulcers display increased plasmin activity that results in increased VEGF degradation and reduced VEGF concentrations (52, 53). Lauer et al. showed that wound fluid from chronic leg ulcers degraded wild type VEGF in vitro but not a recombinant proteolysis resistant VEGF mutant where the plasmin binding site within the HBD was altered (52, 53). This data suggests that blocking protease binding to the HBD on VEGF to control its proteolysis in the chronic wound environment might be a key strategy to improve wound healing, and support the hypothesis that by preventing VEGF degradation by proteases, PR1P may preserve VEGF signaling and critically improve cell survival from tissue injury. The results herein suggest that PR1P protects VEGF from plasmin degradation by binding VEGF on its heparin binding domain (see FIGS. 5 and 6) where plasmin and elastase also bind. The HBD on VEGF is thought to play an important role in determining VEGF isomer receptor specific binding and signaling properties. By preventing VEGF degradation by proteases, PR1P ostensibly preserves VEGF receptor binding and signaling properties. Furthermore, it was recently shown that PR1P enhances VEGF binding to VEGFR-2 and to NRP-1 compared to VEGFR1, suggesting that PR1P binding to VEGF may stabilize VEGF binding features even in the absence of protcasc. Kurtagic et al. found that treatment of VEGF with the protcase clastasc resulted in smaller sized VEGF fragments with altered HBDs that preferentially bound VEGFR1 thus upregulating VEGFR 1 compared to VEGFR-2 signaling (34). In addition, it was found that the elastase generated VEGF fragments had a reduced chemoattractant effect on endothelial cells but increased effect on progenitor cells and macrophages (48) highlighting the notion that altered VEGF fragments have altered binding and signaling properties compared to the original VEGF. Together, these results support the findings outlined herein which suggest that by stabilizing VEGF from protease degradation, and specifically from the effects of proteases on the HBD, PR1P may preserve VEGF signaling through VEGFR-2.

Several studies show that reduced VEGF and VEGFR-2 (8) levels in vivo and in vitro are associated with pulmonary endothelial and epithelial cell apoptosis (20). These and similar findings led many to believe that over-expression of VEGF in the lung could be curative in emphysema (54), however, enthusiasm for this type of therapy is tempered by clinical trials using systemic VEGF therapy (41) that were complicated by systemic and pulmonary toxicities including increased vascular permeability leading to hypotension (55) and tissue and lung edema (56, 57). It was found that PR1P upregulates endogenous VEGF signaling both in vitro and in vivo when delivered directly into the lungs. Although these studies were not designed to evaluate toxicity from drug administration, ill effects were not observed from therapy during the 24-hour experimental period (data not shown). The results outlined herein raise the possibility that a strategy to use PR1P to upregulate endogenous VEGF signaling in vivo may avoid toxicity seen with alternative and more conventional forms of VEGF therapy. In addition, PR1P was effective in-vitro and in-vivo in the absence of co-administration of exogenous VEGF, suggesting that it capably binds endogenous VEGF secreted by the cells in vitro and within large tissues. PR1P could be used to avoid complications associated with constitutive VEGF over-expression or systemic VEGF treatment by stabilizing and potentiating the signaling of local endogenous VEGF. It will be important to clearly delineate the fate and pharmacokinetics of PR1P in evaluating its toxicity potential. Because of its small size, PR1P could also be more easily packaged as cargo inside nanometer or micron sized storage vehicles for tissue or cell targeted therapy.

Finally, the experiments using FACS of lung cells recovered from whole lungs of mice treated with nebulized LPS in the presence or absence of PR1P suggest that the reduction in lung epithelial cell apoptosis from PR1P therapy is a small fraction of the total reduction in total lung cell apoptosis. Because lung epithelial cells comprise only 3-5% of the total lung cell population (58) the data suggests that the effect of PR1P on lung cell apoptosis is primarily on non-epithelial cell types, including native lung cells or cells recruited to the lungs by LPS, such as inflammatory cells (59). Lung endothelial, epithelial and alveolar macrophages all express VEGF receptors to varying degrees (15), as do neutrophils and other inflammatory cells (14), and so the reduction in total cell apoptosis that was detected from PR1P therapy likely includes a subset of these cell populations. Differences in how these cell populations respond to PR1P will provide valuable information necessary to design targeted PR1P based lung cell therapeutics for emphysema and other VEGF dependent lung disorders.

In summary, the studies described herein revealed that PR1P, a novel VEGF binding and angiogenic peptide that was recently designed from sequences within a VEGF binding domain on prominin-1, mitigates cigarette smoke toxin induced epithelial cell apoptosis in vitro, and LPS induced lung cell and specifically epithelial cell apoptosis in vivo in mice via upregulation of endogenous VEGF signaling. Computer modeling and molecular biology studies support the hypothesis that PR1P binds VEGF near its HBD thereby stabilizing VEGF from proteolytic degradation. The implications of the findings outlined herein are that PR1P could play a potential critical role in reducing apoptosis in emphysema or other lung or systemic disorders characterized by VEGF signaling dysregulation by stabilizing and upregulating endogenous VEGF. These studies provide a proof of principle for a novel approach using small targeting peptides to enhance the activity of endogenous VEGF to enhance lung cell and tissue survival from acute or chronic injury. These studies also support a role for short sequence peptides in modulating growth factor signaling in general.

### Materials and Methods

### VEGF and Prominin-1 Cellulose Peptide Arrays

Spot peptide arrays were prepared at the Massachusetts Institute of Technology (MIT) Biopolymers Facility (Cambridge, MA). Each spot in the array was comprised of a bound single 12-mer contiguous peptide whose sequence was determined using a 3-residue offset in order to cover the entire antigen sequence of human VEGF, e.g. spot 1 contained amino acids 1-12 of the original protein, spot 2 contained amino acids 3-15, spot 3 contained amino acids 6-18, etc.). The membranes with bound peptides were pre-incubated with 1× T-TBS blocking buffer for 2 h, washed twice with PBS and then incubated with recombinant human Prominin-1 (Peprotech, Rocky Hill, NJ) at a concentration of 0.5 μg/ml for 24 h in T-TBS blocking buffer. Membranes were subsequently washed three times (10 minutes) with T-TBS, and then incubated with anti-Prominin-1 antibody (Neomarkers, Fremont, CA) in T-TBS blocking buffer (1 μg/ml) for 1 h. Membranes were then washed 3 times with TBS and incubated with peroxidase-labeled anti-mouse IgG (1 μg/ml, Sigma-Aldrich, St. Louis, MO) in T-TBS blocking buffer for 1 h, and washed 3 times (10 min) in T-TBS. Finally, membranes were analyzed for peptide-bound Prominin-1-antibody complexes using chemiluminescence.

### Western Blot Analysis

BEAS-2B cells (2×10⁶, (ATCC, Manassas, VA) were cultured on 10 cm plates coated with fibronectin (0.01 mg/mL, Sigma-Aldrich, St. Louis, MO)) and bovine collagen type I (0.03 mg/mL, Sigma-Aldrich, St. Louis, MO)×2 days in BEGM medium, which includes 10 growth supplements provided by the vendor and added to BEBM (Lonza/Clonetics, Allendale, NJ). Cells were then washed and supplement deprived in BEBM for 24 hours prior to lysis in RIPA buffer (Boston Bio-products, Worchester, MA) supplemented with a phosphatase and protease inhibitor cocktail (cOmplete™, Mini, EDTA-free Protease Inhibitor Cocktail and 1 mM PMSF (both from Sigma-Aldrich, St. Louis, MO)). Insoluble debris were removed by centrifugation (13,000 g) for 10 min at 4° C. Protein concentration was measured using the Bradford protein assay, according to the manufacturer's instructions (Thermo Scientific, Rockland, IL). Proteins were resolved in non-reduced buffer and run on 12% SDS-PAGE and transferred onto nitrocellulose membranes. The membranes were treated with blocking buffer (PBS containing 5% skim milk and 0.1% Tween-20) for 1 h prior to overnight incubation at 4° C. in the same blocking buffer but with primary antibodies. The membranes were then washed with PBS-0.1% Tween-20 and incubated with secondary antibodies for 1 h at RT, washed with PBS and developed using an ECL kit (Thermo Scientific, Rockland, IL).

### Inhalation Studies

8 week old female C3H/HENCrl (Charles River Laboratories, Cambridge, MA) mice (22±2 g) were placed in a whole-animal nebulization chamber (14×5×8 cm) and allowed to spontaneously inhale (20 min) nebulized LPS (Salmonela enterica, Sigma-Aldrich, St. Louis, MO), PR1P, scrambled peptide (SP), or VEGF. Note nebulization solutions were made with 3 ml 0.9% normal saline containing either 0.5 mg/ml LPS, 300 μg/mL (PR1P or SP) or 500 ng/ml (VEGF). Nebulization machine was a Proneb Ultra II Nebulizer (Pari Respiratory Equipment, Midlothian, VA). Animals were sacrificed 24 hours after nebulization and their lungs harvested for lung cell isolation, and lung cells were prepared for FACS analysis as described below (see Lung cell isolation and FACS analysis).

### TUNEL Staining for Microscopy

BEAS-2B cells (1×10⁵) were plated for 24 hours on Collagen I (50 ug/ml, Sigma-Aldrich, St. Louis, MO) coated glass in BEGM containing 0.1% serum, washed three times with PBS and exposed to serum-free BEBM (control) or cigarette smoke extract (CSE) exposed serum-free BEBM (see cigarette smoke treatment) for 24 hours in the presence or absence of 10 μg/ml PR1P. Following CSE exposure, the cells were washed 3 times with PBS, fixed with 2% PFA, and prepared for TUNEL staining to identify cell apoptosis according to manufacturer instructions (Sigma-Aldrich, St. Louis, MO).

### Lung Cell Isolation and FACS Analysis

Lungs were harvested from treated animals, cut into small pieces using surgical scissors and suspended and gently stirred in 3 ml serum free BEBM containing Liberase (2 mg ml, Sigma-Aldrich, St. Louis, MO). Dispase (5 U/mL, Stemcell Technology. Vancouver, Canada) and deoxyribonuclease (DNase, 50 U/mL, Sigma-Aldrich, St. Louis, MO) for 30 minutes at 37° C. The digested tissue was passed through a 70-μm mesh (Thermo Fisher Scientific, Cambridge, MA) and the filtered cells were washed twice with FACS media (PBS containing 3% FCS) and centrifuged at 1000 rpm×5 minutes. The pelleted material was incubated for 90 seconds in erythrocyte-lysis buffer (Sigma-Aldrich, St. Louis, MO.) and resuspended in FACS media. The suspended cells were then fixed with paraformaldehyde (2%, 15 minutes at RT), permeabilized with Tween (0.7%, 15 minutes at room temperature) and incubated with anti-pVEGFR-2 (Cell Signaling, Danvers, MA) to identify phosphorylation of VEGFR-2, and/or with Annexin V (eBioscience, San Diego CA), Caspase 3 or TUNEL (both from Thermo Fisher Scientific, Cambridge, MA) to identify cell apoptosis. In specific experiments, cells were treated with anti-CD326 antibody (BD Biosciences, San Jose, CA) to identify epithelial cells. Cells were subsequently washed with FACS media and analyzed by a flow cytometer (BD Biosciences, San Jose, CA, USA) running FlowJo 7.2.2, software (Tomy Digital Biology. Tokyo, Japan).

### Cell Culture

BEAS-2B human bronchial epithelial cells (ATCC, Manassas, VA) were grown on 10 cm cell culture flasks that were pre-coated with fibroncctin (0.01 mg/mL, Sigma-Aldrich, St. Louis, MO) and bovine collagen type I (0.03 mg/mL, Sigma-Aldrich, St. Louis, MO) in BEBM medium (Lonza/Clonetics, Allendale, NJ). Experiments were performed using cells with less than 10 culture passages.

### Reagents, Peptides and Antibodies

Plasmin was purchased from American Diagnostica (Stamford, CT) and elastase from Fitzgerald Industries (North Acton, MA). The 12-mer peptide, PR1P, and its scrambled form were commercially synthesized by Biomatik (Wilmington, DE). VEGF was purchased from Peprotech (Rocky Hill, NJ). Primary antibodies used to identify VEGF were purchased from Santa Cruz, (Santa Cruz, CA. Primary antibodies used to identify phosphorylated AKT (pAKT) were purchased from Cell Signaling, (Danvers, MA). Primary antibodies used to identify phosphorylated VEGFR-2 (pVEGFR-2) were purchased from R&D Systems, Minneapolis, MN. Primary antibody used to identify β-actin and HRP-conjugated anti-rabbit IgG secondary antibody were purchased from Sigma-Aldrich, St. Louis, MO.

### Statistics

Analysis of statistical significance was performed using Graphpad Prism v6. Groups of 3 or more were analyzed by one-way ANOVA followed by Tukey's post-test for comparison between pairs of groups. Multiple T tests were corrected for using the Holm-Sidak method. Where appropriate, comparisons were made between treatment and matched pair control samples treated on the same day. The non-parametric Wilcoxon signed rank test was used to analyze groups of ratios.

### Example 2: Using the Novel Short Peptide PR1P to Treat Lung Disorder

Previous work (US 20130045922 A1) involves the use of a 12 amino-acid peptide (PR1P) that was engineered to stabilize and was shown to enhance the activity of endogenous vascular endothelial growth factor (VEGF) in wound healing, burns, tissue repair, fertility, myocardial infarction, myocardial hypertrophy, tissue revascularization in stroke, limb ischemia, and peripheral artery disease, in bone repair, tissue grafts and in tissue engineering, as well as in ALS, Multiple Sclerosis, Alzheimer's disease, and Parkinson's disease. The amino acid sequence of PR1P (DRVQRQTTTVVA; SEQ ID NO: 1) is from an extracellular domain of the penta-span trans-membrane glycoprotein, prominin-1. Data in US 20130045922 shows that PR1P a) binds VEGF, b) enhances VEGF binding to endothelial cells and c) potentiates VEGF angiogenic activity in-vivo (hind limb ischemia and myocardial infarction models) as well as improves neuron cell growth in vitro. Because VEGF levels in the lungs are curiously 500 fold greater than in serum and because many lung disorders are characterized by reduced or dysregulated VEGF signaling and increased apoptosis, it is hypothesized that PR1P would be effective in treating lung injury from multiple causes.

The data shown herein is based on results obtained from an in vitro lung emphysema model whereby PR1P was shown to mitigate the effects of cigarette smoke toxicity on respiratory airway epithelial cells. Emphysema, defined pathologically as abnormal enlargement of air spaces distal to the terminal bronchioles, is accompanied by the destruction of alveolar walls without fibrosis, and is a major component of chronic obstructive pulmonary disease (COPD), a syndrome that affects nearly 5% of the world population. COPD is predicted to become the fourth leading cause of death worldwide by 2030, and so due to an aging world population and increasing number of smokers, the burden of medical and social resources for COPD is estimated to rise to $47 trillion worldwide by 2030.

Disease progression in emphysema is thought to be due to increased apoptosis of lung cells associated with reduced VEGF activity that is critical for lung homeostasis. Despite this correlation in the lungs, enthusiasm for using VEGF therapy in vivo has generally been tempered by VEGF's short half-life necessitating frequent drug administration, as well as significant lung and systemic toxicity from its systemic administration. Developing a drug capable of targeting and upregulating endogenous VEGF in the diseased lung microenvironment could potentially help patients with lung disorders of multiple causes without inducing local or systemic toxicity.

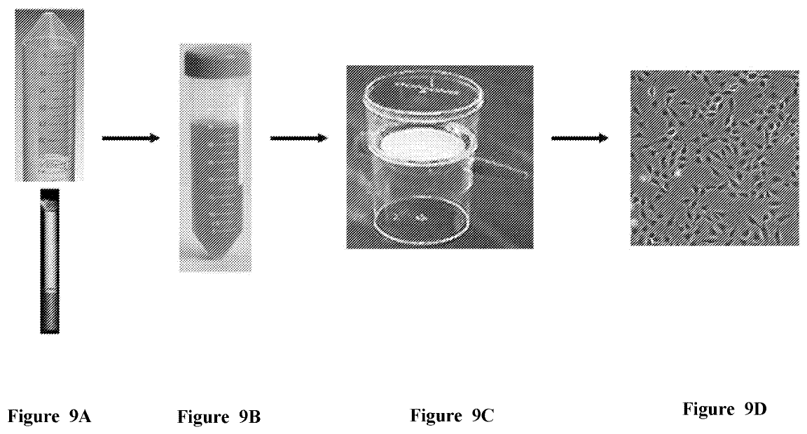

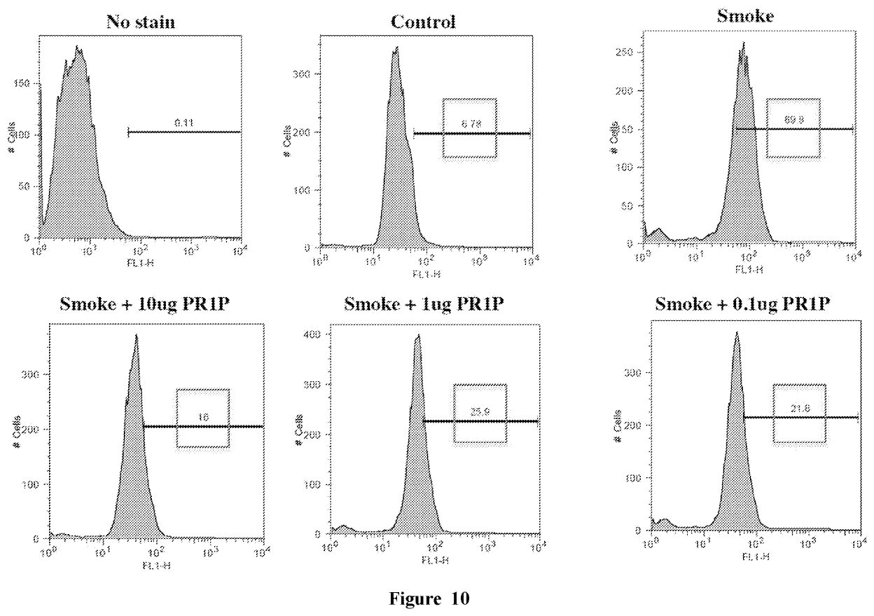

Based on recent work, it is believed that PR1P could be delivered directly into the lungs in humans (by instillation or inhalation) or systemically to target and upregulate endogenous VEGF in the lungs, and thus could be used specifically in emphysema, but also for other lung disorders as well, to reduce lung cell apoptosis and enhance lung tissue remodeling. It was recently established an in vitro lung emphysema model to monitor cigarette smoke toxicity on bronchial epithelial lung cells (BEAS-2B). In the model described herein, BEAS-2B cells are exposed to cigarette smoke (CS) treated media (FIGS. 9A-9D) for 24 hours in the presence and absence of PR1P. First, it was determined that exposure of cells to smoke treated media arrested cell proliferation which was not mitigated by PR1P (FIG. 7). However, analysis of phase images captured of the cells at 0 and 24 h after exposure to smoke treated media in the absence and presence of PR1P showed that cigarette smoke induced an increase in the proportion of a round cell phenotype compared to a flat cell phenotype (see FIG. 2A) which was mitigated by the presence of PR1P (FIGS. 2A, 2C, and 2D). Analysis of fluorescence images of BEAS-2B cells exposed to cigarette smoke treated media in the absence and presence of PR1P and stained with TUNEL revealed that the cells with round phenotype were all positively stained with TUNEL, suggesting they were apoptotic (FIG. 2E). FACS analysis using TUNEL staining of BEAS-2B cells exposed to cigarette treated media in the presence and absence of PR1P showed that cigarette smoke induced an increase in the proportion of apoptotic cells that was mitigated by PR1P (FIG. 10). Note the similarities in proportions of round cells in FIGS. 2C and 2D and the percentage of apoptotic cells determined by FACS in FIG. 10. Together, the data outlined herein suggests that PR1P protects BEAS-2B cells from the toxic effects of cigarette smoke in vitro. It is hypothesized that PR1P could be used to protect respiratory epithelial cells in vivo by minimizing apoptosis and improving cell survival following exposure to cigarette smoke and other environmental toxins, or due to acute or chronic lung injury from multiple causes. It is envisioned that the product will be delivered directly into the lung (intratracheal instillation or inhalation) or given systemically (intravenously) and it is expected that testing of specificity, safety, and efficacy will be necessary in mice and larger animals prior to its development for clinical use in humans.

### Example 3. PR1P Treats Emphysema

There are no medicines that treat or prevent emphysema, a devastating chronic progressive disease characterized by chronic destruction of the alveolar walls that leads to enlargement of the alveoli and reduction of the lung's gas exchange capacity. Current emphysema therapies target disease symptomatology including lung inflammation, wheezing and infection, and are not directed at disease etiology.

The PR1P therapy described herein targets and upregulates endogenous VEGF signaling, thereby enhancing lung remodeling to relieve disease burden. The experiments described herein were designed to evaluate whether inhaled PR1P could promote lung repair in a murine elastase induced emphysema model and to further characterizing the PR1P mechanism of action. The data in mice suggest that PR1P mitigates elastase induced murine emphysema in both a therapeutic and preventive fashion. In addition, in vitro studies revealed that the VEGF receptor-2 (VEGFR2) inhibitor SU5416 significantly reduced PR1P induced VEGFR2 phosphorylation as well as downstream VEGF signaling (phosphorylation of AKT), suggesting that PR1P action requires VEGFR2 activation.

To characterize the ability of inhaled PR1P to delay onset of, or reverse pathology in emphysema, an established murine emphysema model was used, in which emphysema phenotype was induced by intra-tracheal injection of porcine pancreatic elastase (PPE). To establish a timeline of lung injury in the model, C3H/HENCrl adult mice were treated with intra-tracheal Elastase (0.1 U in 50 μl PBS) or with equivalent volume of PBS and the degree of emphysema phenotype (lung injury) was assessed by monitoring lung histology at 24 h, 48 h, 72 h and 96 h following treatment. Consistent with previously reported reports. PPE induced characteristic changes in lung architecture was observed, such as destruction of the alveolar walls and alveoli enlargement as early as 24 h after injection (FIGS. 11 and references 61, 62) that was indistinguishable from injury seen at 4 days.

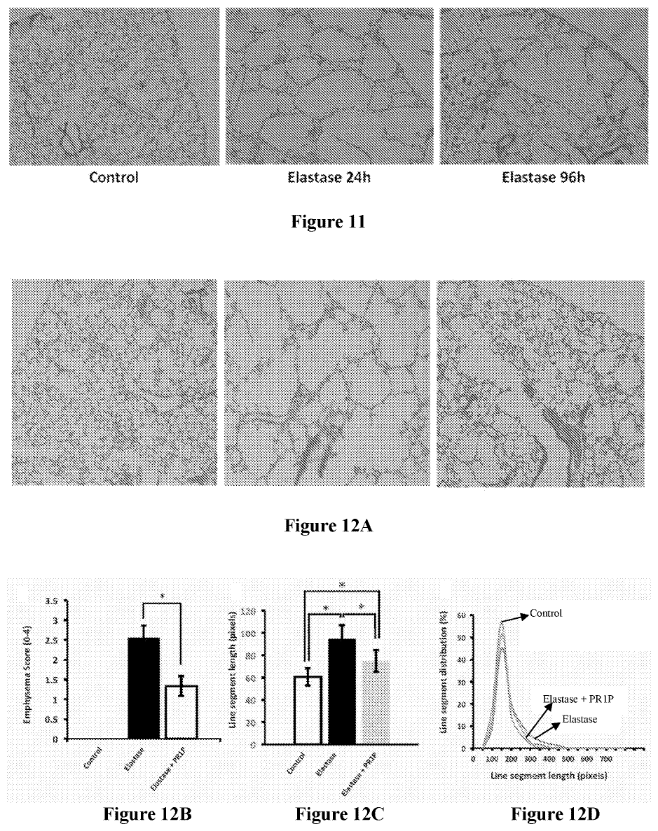

To determine if PR1P therapy could be used to delay the onset of disease in this model, or even begin to induce lung repair or remodeling, mice exposed to intra-tracheal elastase were treated with inhaled PR1P (1 mg/ml, over 20 minutes, daily) or control vehicle beginning at 24 hours after elastase treatment. Importantly, the half-life of the exogenous elastase administered into the animal lungs was short (approximately 50 minutes³), and single doses of elastase inhibitors were effective in mitigating the elastase induced injury only when given immediately before or within 4-8 hours after elastase administration^{63,64}. Thus, elastase is required to initiate the process of connective tissue destruction, but disease progression is due to ongoing inflammation and lung remodeling in the absence of active exogenous elastase. The results strongly suggests that PR1P treatment at 24 h following elastase treatment would not directly interfere with the ability of elastase to initiate lung injury. Qualitative scoring of histological sections of the lungs at 4 days by a histologist blinded to treatment indicated that PR1P given daily by inhalation starting at 24 h significantly improved lung architecture (FIGS. 12A and 12B) Blinded quantitative analysis of the same slides using an established method to grade lung tissue density⁵ also indicated that PR1P significantly reversed damage that had already developed within 24 h of elastase administration (FIGS. 12C and 12D). Together, these findings imply that PR1P has the potential to be used as both a therapeutic as well as a preventive therapy for emphysema.

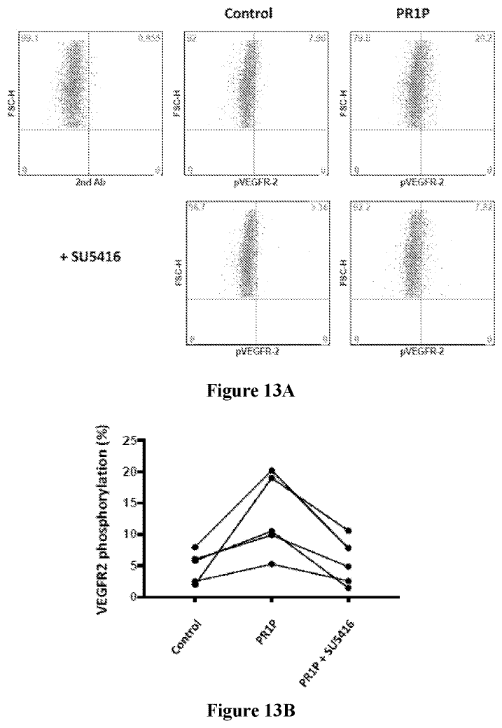

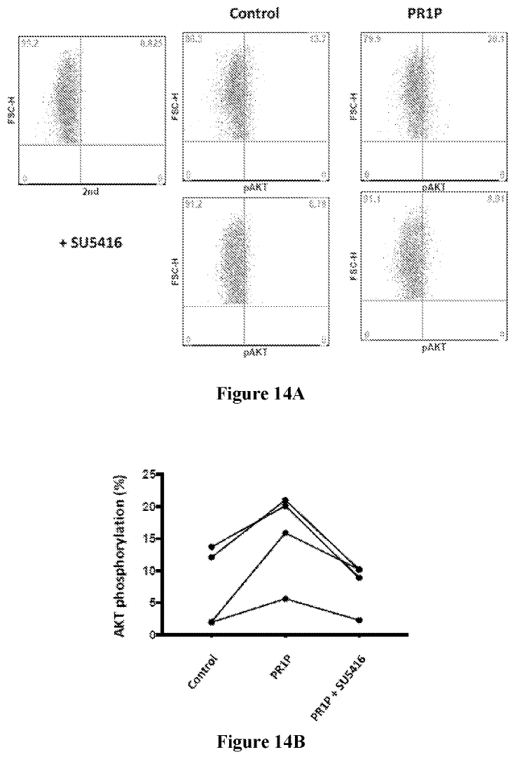

It was previously shown in part that PR1P binds VEGF, increases VEGF binding to VEGF receptors, and induces downstream VEGF signaling including the phosphorylation of VEGF receptor 2 and AKT. To determine whether PR1P mediation of VEGF signaling requires VEGFR2 activation, lung epithelial cells (Beas-2B) were treated with the specific VEGFR2 inhibitor SU5416 (20 mM) or with vehicle alone as control for 24 hours and then exposed PR1P (125 μM) or vehicle for 20 min. Cells were then harvested and levels of phosphorylated VEGFR2 and AKT were assessed using FACS. FIGS. 13A-14B show that SU5416 significantly reduced the effect of PR1P on the phosphorylation levels of both VEGFR-2 and AKT. These findings strongly support the notion that PR1P upregulation of VEGF signaling requires VEGFR-2 activation.

### Methods

### Intra-Tracheal Elastase Administration

Mice were anesthetized with a mixture of ketamine (100 mg/kg) and xylazine (15 mg/kg) via intraperitoneal injection, and were orally intubated with a 22G plastic angiocatheter (Becton Dickinson and Company, Franklin Lakes, NJ) through which a single dose of porcine pancreatic elastase (PPE, 0.1 enzymatic activity units (U) dissolved in 50 μl sterile normal saline (NS)) or NS alone were delivered. Mice were then allowed to recover from anesthesia and were returned to their cages.

### Mean Linear Intercept (Lm)

Lung tissue sections were prepared from nine animals per group and were stained with hematoxylin and eosin (H&E). Images capturing an entire cross section of each stained animal lungs were stored and assigned random labels. The mean linear intercept, an index widely used to quantify emphysema phenotype⁵, was quantified for a select number of randomly chosen images from each group. Following tissue section analyses, true image labels were revealed and averages of mean linear intercepts were calculated for each treatment group.

### Detection of PR1P Effect on AKT and VEGFR-2 Phosphorylation

Serum-starved monolayers of Beas-2B cells were incubated in the presence or absence of SU5416 (20 μM) for 24 h on 10 cm tissue culture plates. After single washing with PBS, the cells were treated with 3 ml of starvation media in the presence of PR1P (125 μg/ml) or vehicle as control for 20 minutes at 37° C. At the conclusion of the experiments, cells were prepared for FACS analysis using standard procedures. Cells were analyzed for levels of phosphorylated VEGFR2 and AKT using anti pAKT and pVEGFR2 antibodies (Cell signaling, Danvers MA).

### REFERENCES

1. P. J. Barnes, New anti-inflammatory targets for chronic obstructive pulmonary disease. Nat Rev Drug Discov 12, 543-559 (2013).
2. J. Vestbo et al., Global strategy for the diagnosis, management, and prevention of chronic obstructive pulmonary disease: GOLD executive summary. Am J Respir Crit Care Med 187, 347-365 (2013).
3. L. Segura-Valdez et al., Upregulation of gelatinases A and B, collagenases 1 and 2, and increased parenchymal cell death in COPD. Chest 117, 684-694 (2000).
4. T. Suzuki et al., Proteinase-activated receptor-1 mediates elastase-induced apoptosis of human lung epithelial cells. Am J Respir Cell Mol Biol 33, 231-247 (2005).
5. K. Ohnishi, M. Takagi, Y. Kurokawa, S. Satomi, Y. T. Konttinen, Matrix metalloproteinase-mediated extracellular matrix protein degradation in human pulmonary emphysema. Lab Invest 78, 1077-1087 (1998).
6. H. Kanazawa, Role of vascular endothelial growth factor in the pathogenesis of chronic obstructive pulmonary disease. Med Sci Monit 13, RA189-195 (2007).
7. J. A. Marwick et al., Cigarette smoke regulates VEGFR2-mediated survival signaling in rat lungs. J Inflamm (Lond) 7, 11 (2010).
8. Y. Kasahara et al., Inhibition of VEGF receptors causes lung cell apoptosis and emphysema. J Clin Invest 106, 1311-1319 (2000).
9. N. Yokohori, K. Aoshiba, A. Nagai, J. Respiratory Failure Research Group in, Increased levels of cell death and proliferation in alveolar wall cells in patients with pulmonary emphysema. Chest 125, 626-632 (2004).
10. M. Plataki et al., Apoptotic mechanisms in the pathogenesis of COPD. Int J Chron Obstruct Pulmon Dis 1, 161-171 (2006).
11. H. Koh et al., Protective role of vascular endothelial growth factor in endotoxin-induced acute lung injury in mice. Respir Res 8, 60 (2007).
12. R. J. Kaner, R. G. Crystal, Compartmentalization of vascular endothelial growth factor to the epithelial surface of the human lung. Mol Med 7, 240-246 (2001).
13. N. Ferrara, H. P. Gerber, J. LeCouter, The biology of VEGF and its receptors. Nat Med 9, 669-676 (2003).
14. A. I. Papaioannou, K. Kostikas, P. Kollia, K. I. Gourgoulianis, Clinical implications for vascular endothelial growth factor in the lung: friend or foe? Respir Res 7, 128 (2006).
15. M. Mura, C. C. dos Santos, D. Stewart, M. Liu, Vascular endothelial growth factor and related molecules in acute lung injury. J Appl Physiol (1985) 97, 1605-1617 (2004).
16. N. Ferrara, Vascular endothelial growth factor: basic science and clinical progress. Endocrine reviews 25, 581-611 (2004).
17. D. R. Senger, C. A. Perruzzi, J. Feder, H. F. Dvorak, A highly conserved vascular permeability factor secreted by a variety of human and rodent tumor cell lines. Cancer research 46, 5629-5632 (1986).
18. D. R. Thickett, L. Armstrong. A. B. Millar, A role for vascular endothelial growth factor in acute and resolving lung injury. Am J Respir Crit Care Med 166, 1332-1337 (2002).
19. N. F. Voelkel, J. Gomez-Arroyo, S. Mizuno, COPD/emphysema: The vascular story. Pulm Circ 1, 320-326 (2011).
20. K. Tang, H. B. Rossiter, P. D. Wagner, E. C. Breen, Lung-targeted VEGF inactivation leads to an emphysema phenotype in mice. J Appl Physiol (1985) 97, 1559-1566; discussion 1549 (2004).
21. J. A. Marwick et al., Cigarette smoke disrupts VEGF165-VEGFR-2 receptor signaling complex in rat lungs and patients with COPD: morphological impact of VEGFR-2 inhibition. Am J Physiol Lung Cell Mol Physiol 290, L897-908 (2006).
22. Y. Kasahara et al., Endothelial cell death and decreased expression of vascular endothelial growth factor and vascular endothelial growth factor receptor 2 in emphysema. Am J Respir Crit Care Med 163, 737-744 (2001).
23. H. P. Gerber et al., Vascular endothelial growth factor regulates endothelial cell survival through the phosphatidylinositol 3′-kinase/Akt signal transduction pathway. Requirement for Flk-1/KDR activation. J Biol Chem 273, 30336-30343 (1998).
24. H. P. Gerber, V. Dixit, N. Ferrara, Vascular endothelial growth factor induces expression of the antiapoptotic proteins Bcl-2 and A1 in vascular endothelial cells. J Biol Chem 273, 13313-13316 (1998).
25. X. X. Lin et al., Cigarette smoke extract-induced BEAS-2B cell apoptosis and anti-oxidative Nrf-2 up-regulation are mediated by ROS-stimulated p38 activation. Toxicol Mech Methods 24, 575-583 (2014).
26. I. Edirisinghe, I. Rahman, Cigarette smoke-mediated oxidative stress, shear stress, and endothelial dysfunction: role of VEGFR2. Ann N Y Acad Sci 1203, 66-72 (2010).
27. T. D. Nauta, V. W, van Hinsbergh. P. Koolwijk, Hypoxic signaling during tissue repair and regencrative medicine. Int J Mol Sci 15. 19791-19815 (2014).
28. Z. Taimeh, J. Loughran, E. J. Birks, R. Bolli, Vascular endothelial growth factor in heart failure. Nat Rev Cardiol 10, 519-530 (2013).
29. Z. Raval, D. W. Losordo, Cell therapy of peripheral arterial disease: from experimental findings to clinical trials. Circ Res 112, 1288-1302 (2013).
30. Y. Abadie et al., Decreased VEGF concentration in lung tissue and vascular injury during ARDS. Eur Respir J 25, 139-146 (2005).
31. J. A. Clayton, D. Chalothorn, J. E. Faber. Vascular endothelial growth factor-A specifies formation of native collaterals and regulates collateral growth in ischemia. Circ Res 103. 1027-1036 (2008).
32. C. G. Lee et al., Studies of vascular endothelial growth factor in asthma and chronic obstructive pulmonary disease. Proc Am Thorac Soc 8, 512-515 (2011).
33. H. G. Moon et al., CCN1 secretion and cleavage regulate the lung epithelial cell functions after cigarette smoke. Am J Physiol Lung Cell Mol Physiol 307, L326-337 (2014).
34. E. Kurtagic, M. P. Jedrychowski, M. A. Nugent, Neutrophil elastase cleaves VEGF to generate a VEGF fragment with altered activity. Am J Physiol Lung Cell Mol Physiol 296. L534-546 (2009).
35. A. Adini et al., The stem cell marker prominin-1/CD133 interacts with vascular endothelial growth factor and potentiates its action. Angiogenesis 16, 405-416 (2013).
36. A. Adini et al., A novel strategy to enhance angiogenesis in vivo using the small VEGF-binding peptide PR1P. Angiogenesis, (2017).
37. L. Larsson et al., Identification of bacterial and fungal components in tobacco and tobacco smoke. Tob Induc Dis 4, 4 (2008).
38. R. Z'Graggen B et al., Acute lung injury: apoptosis in effector and target cells of the upper and lower airway compartment. Clin Exp Immunol 161, 324-331 (2010).
39. B. A. Keyt et al., Identification of vascular endothelial growth factor determinants for binding KDR and FLT-1 receptors. Generation of receptor-selective VEGF variants by site-directed mutagenesis. J Biol Chem 271, 5638-5646 (1996).
40. B. A. Keyt et al., The carboxyl-terminal domain (111-165) of vascular endothelial growth factor is critical for its mitogenic potency. J Biol Chem 271, 7788-7795 (1996).
41. M. Giacca. S. Zacchigna. VEGF gene therapy: therapeutic angiogenesis in the clinic and beyond. Gene Ther 19, 622-629 (2012).
42. T. Arcondeguy, E. Lacazette, S. Millevoi, H. Prats, C. Touriol, VEGF-A mRNA processing, stability and translation: a paradigm for intricate regulation of gene expression at the post-transcriptional level. Nucleic Acids Res 41, 7997-8010 (2013).
43. G. Pages, J. Pouyssegur, Transcriptional regulation of the Vascular Endothelial Growth Factor gene--a concert of activating factors. Cardiovasc Res 65, 564-573 (2005).
44. P. Bock, K. Gorgas, Fine structure of baroreceptor terminals in the carotid sinus of guinea pigs and mice. Cell Tissue Res 170, 95-112 (1976).
45. I. Stein et al., Translation of vascular endothelial growth factor mRNA by internal ribosome entry: implications for translation under hypoxia. Mol Cell Biol 18, 3112-3119 (1998).
46. V. B. Kumar, S. Binu, S. J. Soumya, H. K, P. R. Sudhakaran, Regulation of vascular endothelial growth factor by metabolic context of the cell. Glycoconj J 31, 427-434 (2014).
47. A. J. Potgens et al., Covalent dimerization of vascular permeability factor/vascular endothelial growth factor is essential for its biological activity. Evidence from Cys to Ser mutations. J Biol Chem 269, 32879-32885 (1994).
48. E. Kurtagic, C. B. Rich, J. A. Buczek-Thomas, M. A. Nugent, Neutrophil Elastase-Generated Fragment of Vascular Endothelial Growth Factor-A Stimulates Macrophage and Endothelial Progenitor Cell Migration. PLoS One 10, e0145115 (2015).
49. L. Vermeulen, G. De Wilde, P. Van Damme, W. Vanden Berghe, G. Haegeman, Transcriptional activation of the NF-kappaB p65 subunit by mitogen-and stress-activated protein kinase-1 (MSK1). Embo J 22, 1313-1324 (2003).
50. C. M. Greene, N. G. McElvaney, Proteases and antiproteases in chronic neutrophilic lung disease-relevance to drug discovery. Br J Pharmacol 158, 1048-1058 (2009).
51. R. T. Abboud, S. Vimalanathan, Pathogenesis of COPD. Part I. The role of protease-antiprotease imbalance in emphysema. Int J Tuberc Lung Dis 12, 361-367 (2008).
52. G. Lauer et al., Expression and proteolysis of vascular endothelial growth factor is increased in chronic wounds. J Invest Dermatol 115, 12-18 (2000).
53. G. Lauer, S. Sollberg, M. Cole, T. Krieg, S. A. Eming, Generation of a novel proteolysis resistant vascular endothelial growth factor165 variant by a site-directed mutation at the plasmin sensitive cleavage site. FEBS Lett 531, 309-313 (2002).
54. W. I, de Boer, H. Yao, I. Rahman, Future therapeutic treatment of COPD: struggle between oxidants and cytokines. Int J Chron Obstruct Pulmon Dis 2, 205-228 (2007).
55. M. D. Hariawala et al., VEGF improves myocardial blood flow but produces EDRF-mediated hypotension in porcine hearts. J Surg Res 63, 77-82 (1996).
56. I. Baumgartner et al., Lower-extremity edema associated with gene transfer of naked DNA encoding vascular endothelial growth factor. Ann Intern Med 132, 880-884 (2000).
57. S. Yla-Herttuala. T. T. Rissanen, I. Vajanto, J. Hartikainen, Vascular endothelial growth factors: biology and current status of clinical applications in cardiovascular medicine. J Am Coll Cardiol 49, 1015-1026 (2007).
58. B. D. Singer et al., Flow-cytometric method for simultaneous analysis of mouse lung epithelial, endothelial, and hematopoietic lineage cells. Am J Physiol Lung Cell Mol Physiol, ajplung 00334 02015 (2016).
59. T. Tschernig, K. S. Janardhan, R. Pabst, B. Singh, Lipopolysaccharide induced inflammation in the perivascular space in lungs. J Occup Med Toxicol 3. 17 (2008).
60. E. Stuttfeld, K. Ballmer-Hofer, Structure and function of VEGF receptors. IUBMB Life 61, 915-922 (2009).
61. Hellbach K, Meinel F G, Conlon T M, et al. X-Ray Dark-field Imaging to Depict Acute Lung Inflammation in Mice. Sci Rep 2018;8:2096.
62. Munoz-Barrutia A, Ceresa M, Artaechevarria X, Montuenga L M, Ortiz-de-Solorzano C. Quantification of lung damage in an elastase-induced mouse model of emphysema. Int J Biomed Imaging 2012;2012:734734.
63. Stone P J, Lucey E C, Calore J D, et al. The moderation of elastase-induced emphysema in the hamster by intratracheal pretreatment or post-treatment with succinyl alanyl alanyl prolyl valine chloromethyl ketone. Am Rev Respir Dis 1981; 124:56-9.
64. Gudapaty S R, Liener I E, Hoidal J R, Padmanabhan R V, Niewoehner D E, Abel J. The prevention of elastase-induced emphysema in hamsters by the intratracheal administration of a synthetic elastase inhibitor bound to albumin microspheres. Am Rev Respir Dis 1985;132:159-63.

65. Knudsen L, Weibel E R, Gundersen H J, Weinstein F V, Ochs M. Assessment of air space size characteristics by intercept (chord) measurement: an accurate and efficient stereological approach. J Appl Physiol (1985) 2010; 108:412-21.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one. or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations. and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

1. A method of treating a lung disorder associated with dysregulated VEGF signaling, the method comprising administering to a subject in need thereof an effective amount of a peptide comprising an amino acid sequence that is at least 80% or at least 90% identical to the amino acid sequence of DRVQRQTTTVVA (SEQ ID NO: 1).

2. The method of claim 1, wherein the peptide is no more than 50 amino acids in length.

3. The method of claim 1, wherein the peptide comprises the amino acid sequence of SEQ ID NO: 1.

4. (canceled)

5. The method of claim 1, wherein the peptide comprises an amino acid sequence that has one or more conservative amino acid substitutions in SEQ ID NO: 1.

6. The method of claim 1, wherein the peptide is cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combinations thereof.

7. The method of claim 1, wherein the peptide further comprises a fusion domain.

8. The method of claim 7, wherein the fusion domain is selected from the group consisting of polyhistidine, Glu-Glu, glutathione S transferase (GST), thioredoxin, protein A, protein G, an immunoglobulin heavy chain constant region (Fc), maltose binding protein (MBP), and human serum albumin.

9. (canceled)

10. The method of claim 1, wherein the peptide is a dimer, trimer, tetramer, or pentamer.

11. The method of claim 1, wherein the peptide is attached to a polymer.

12. The method of claim 11, wherein the polymer prolongs serum half-life of the peptide.

13. The method of claim 11, wherein the polymer prolongs shelf-life of the peptide.

14. The method of claim 1, wherein the peptide is a cyclic peptide.

15. The method of claim 1, wherein the peptide is formulated in a pharmaceutical composition.

16-20. (canceled)

21. The method of claim 1, wherein the lung disorder is selected from the group consisting of: severe progressive pulmonary hypertension (PH), neonatal respiratory distress syndrome (RDS), scleroderma with interstitial lung disease, ARDS, COPD, emphysema and bronchopulmonary dysplasia (BPD).

22. The method of claim 1, wherein the lung disorder is associated with cigarette smoke, caused by LPS, or associated with acute or chronic lung injury.

23-24. (canceled)

25. The method of claim 1, wherein the lung disorder is emphysema.

26. The method of claim 1, wherein the lung disorder is chronic obstructive pulmonary disease (COPD).

27-31. (canceled)

32. The method of claim 1, further comprising administering a second agent to the subject in need thereof for the treatment of the lung disorder.

33. The method of claim 1, wherein the subject in need thereof is a mammal.

34-36. (canceled)

37. A peptide comprising an amino acid sequence that is at least 80% or at least 90% identical to the amino acid sequence of DRVQRQTTTVVA (SEQ ID NO: 1), for use in the manufacturing of a medicament for treating a lung disorder associated with dysregulated VEGF signaling.


SEQUENCE LISTING (8 sequences)

SEQ ID NO: 1 - protein, 12 aa, synthetic construct
  REGION 1..12: note = Synthetic polynucleotide
DRVQRQTTTVVA

SEQ ID NO: 2 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 3 - protein, 12 aa, synthetic construct
  REGION 1..12: note = Synthetic polypeptide
KKDRARQENPCG

SEQ ID NO: 4 - protein, 12 aa, synthetic construct
  REGION 1..12: note = Synthetic polypeptide
RARQENPCGPCS

SEQ ID NO: 5 - protein, 12 aa, synthetic construct
  REGION 1..12: note = Synthetic polypeptide
ERRKHLVQDPQT

SEQ ID NO: 6 - protein, 12 aa, synthetic construct
  REGION 1..12: note = Synthetic polypeptide
KARQLELNERTC

SEQ ID NO: 7 - protein, 58 aa, synthetic construct
  REGION 1..58: note = Synthetic polypeptide
KKDRARQENPCGPCSERRKHLVQDPQTCKCSCKNTDSRCKARQLELNERTCRCDKPRR

SEQ ID NO: 8 - protein, 12 aa, synthetic construct
  REGION 1..12: note = Synthetic polypeptide
ARVQRQTTTVVA